(19) United States
(12) Patent Application Publication
(10) Pub. No.: US 2023/0072403 A1
(43) Pub. Date: Mar. 9, 2023

(54) SYSTEMS AND METHODS FOR STROKE CARE MANAGEMENT

(71) Applicant: Imperative Care, Inc., Campbell, CA (US)

(72) Inventors: Michael Strasser, Corte Madera, CA (US); Kirsten Carroll, San Francisco, CA (US); Ramin Rasoulian, Los Angeles, CA (US); Arun Iyengar, Yorktown Heights, NY (US); Leo Kopelow, Winnipeg (CA); Sangshik Park, San Francisco, CA (US)

(21) Appl. No.: 17/822,412

(22) Filed: Aug. 25, 2022

Related U.S. Application Data
(60) Provisional application No. 63/236,876, filed on Aug. 25, 2021.

(51) Int. Cl. G16H 40/67; G16H 10/20

(52) U.S. Cl. CPC G16H 40/67; G16H 10/20

(57) ABSTRACT

Devices, systems, and methods for generating and updating one or more user interfaces of a mobile software application operating on a smart phone of a stroke survivor having an impairment. The method can include tracking a first state of stroke survivor post discharge. The method can also include generating a first user interface configured to be displayed on the smart phone and is dynamically updated based on the tracked first state of the stroke survivor. Further, the method can include a first ribbon in the first user interface corresponding to a first learning content. Moreover, the method can include splitting the learning content based on the impairment associated with the stroke survivor and a first property of the first learning content. Lastly, the method can include a second ribbon in the first user interface corresponding to a second learning content.

### CROSS-REFERENCE TO RELATED APPLICATIONS

This application is related to International Patent Application PCT/US2020/055604, filed Oct. 14, 2020, and claims the benefit of U.S. Provisional Application No. 63/236,876, filed Aug. 25, 2021, entitled “SYSTEMS AND METHODS FOR STROKE CARE MANAGEMENT,” the contents of which are herein incorporated by reference in their entirety.

### INCORPORATION BY REFERENCE

All publications and patent applications mentioned in this specification are herein incorporated by reference in their entirety, as if each individual publication or patent application was specifically and individually indicated to be incorporated by reference in its entirety.

### TECHNICAL FIELD

This disclosure relates generally to the field of post health event care, and more specifically to the field of post-acute stroke management. Described herein are systems and methods of stroke care management.

### BACKGROUND

Stroke is the third most common cause of death in the United States and the most disabling neurologic disorder. Approximately 800,000 patients suffer from stroke annually, and there are about 6 to 8 million stroke survivors. Stroke is a medical emergency characterized by the acute onset of a neurological deficit that persists for at least 24 hours, reflecting focal involvement of the central nervous system, and is the result of a disturbance of the cerebral circulation. Its incidence increases with age. Risk factors for stroke include systolic or diastolic hypertension, hypercholesterolemia, cigarette smoking, heavy alcohol consumption, diabetes, and oral contraceptive use.

Hemorrhagic stroke accounts for about 13% of the annual stroke population. Hemorrhagic stroke often occurs due to rupture of an aneurysm or arteriovenous malformation bleeding into the brain tissue, resulting in cerebral infarction. The remaining 87% of the stroke population are ischemic strokes and are caused by occluded vessels that deprive the brain of oxygen-carrying blood. Ischemic strokes are often caused by emboli or pieces of thrombotic tissue that have dislodged from other body sites or from the cerebral vessels themselves to occlude in the narrow cerebral arteries more distally. When a patient presents with neurological symptoms and signs which resolve completely within 1 hour, the term transient ischemic attack (TIA) is used. Etiologically, TIA and stroke share the same pathophysiologic mechanisms and thus represent a continuum based on persistence of symptoms and extent of ischemic insult.

Notwithstanding the foregoing, once the patient is discharged from the hospital, the patient's road to recovery is long and arduous, especially since existing therapies are incomplete at treating or reversing the effects of the stroke. Many disabilities or effects of the stroke may present early or may not present until days or weeks or months later. As such, patients and their caregivers and/or care partners require many resources, tools, and support in adapting to their current state, recovering after the stroke event, and connecting with their doctor, care network, and other survivors. Accordingly, there exists a need for improved stroke care management after a stroke event.

### SUMMARY

Disclosed herein is a method for generating and updating one or more user interfaces of a mobile software application operating on a smart phone of a stroke survivor having an impairment. In some implementations, the method comprises: tracking, from a plurality of network-based non-transitory storage devices, a first state of stroke survivor post discharge from a hospital; generating a first user interface configured to be displayed on the smart phone, wherein the first user interface is dynamically updated based on the tracked first state of the stroke survivor; including a first ribbon in the first user interface, said first ribbon corresponding to a first learning content enabled on the first user interface based on the first tracked state; determining a plurality of first learning content user interfaces to split the learning content based on the impairment associated with the stroke survivor and a first property of the first learning content; and including a second ribbon in the first user interface, said second ribbon corresponding to a second learning content, wherein the second ribbon is disabled until the plurality of first learning content user interfaces are viewed by the stroke survivor.

In some implementations, the method further includes storing one or more metrics corresponding to the stroke survivor after one or more first learning content user interfaces are viewed by the stroke survivor, wherein the one or more metrics are stored in relation with a first electronic identification corresponding to the first learning content. In some implementations, the method includes further includes generating analysis based on the stored one or more metrics corresponding to a plurality of stroke survivors for the first learning content. In some implementations, the method further includes changing a timing of delivery of the first learning content to the mobile software application based on the generated analysis. In some implementations, the one or more metrics comprises a compliance measurement. In some implementations, the method the one or more metrics comprise a fall detection event. In some implementations, the one or metrics comprise an indication of infection.

In some implementations, a first electronic identification is associated with the learning content. In some implementations, the method further includes removing the learning content from the mobile software application based on the first electronic identification. In some implementations, the method further includes providing a dashboard user interface, said dashboard interface comprising a plurality of tabs; and including a notification indicator adjacent to one of the plurality of tabs, said notification indicator corresponding to an activity detected from the mobile software application. In some implementations, the method further includes updating the first learning content based on one or more scores measured from an assessment. In some implementations, the method further includes generating a suggested list of questions based on the first state prior to an appointment with a health care team member. In some implementations, the method further includes providing an ability to digitally record an answer from the appointment. In some implementations, the method further includes enabling for a plurality of users, from a web interface, an ability to add ideas for a plurality of learning content, write a second learning content, review the second learning content, and send the second learning content to the mobile software application, without requiring the plurality of users to download any local copies corresponding to the learning content and without requiring opening of separate software applications.

Disclosed herein is a system for generating and updating one or more user interfaces of a mobile software application operating on a smart phone of a stroke survivor having an impairment. In some implementations, the system comprises one or more hardware processors configured to: track, from a plurality of network-based non-transitory storage devices, a first state of stroke survivor post discharge from a hospital; generate a first user interface configured to be displayed on the smart phone, wherein the first user interface is dynamically updated based on the tracked first state of the stroke survivor; include a first ribbon in the first user interface, said first ribbon corresponding to a first learning content enabled on the first user interface based on the first tracked state; determine a plurality of first learning content user interfaces to split the learning content based on the impairment associated with the stroke survivor and a first property of the first learning content; and include a second ribbon in the first user interface, said second ribbon corresponding to a second learning content, wherein the second ribbon is disabled until the plurality of first learning content user interfaces are viewed by the stroke survivor.

In some implementations, the one or more hardware processors are further configured to store one or more metrics corresponding to the stroke survivor after one or more first learning content user interfaces are viewed by the stroke survivor, wherein the one or more metrics are stored in relation with a first electronic identification corresponding to the first learning content. In some implementations, the one or more hardware processors are further configured to generate analysis based on the stored one or more metrics corresponding to a plurality of stroke survivors for the first learning content. In some implementations, the one or more hardware processors are further configured to change a timing of delivery of the first learning content to the mobile software application based on the generated analysis. In some implementations, the one or more metrics comprises a compliance measurement. In some implementations, the one or more hardware processors are further configured to enable for a plurality of users, from a web interface, an ability to add ideas for a plurality of learning content, write a second learning content, review the second learning content, and send the second learning content to the mobile software application, without requiring the plurality of users to download any local copies corresponding to the learning content and without requiring opening of separate software applications.

For purposes of summarizing the disclosure, certain aspects, advantages, and novel features are discussed herein. It is to be understood that not necessarily all such aspects, advantages, or features will be embodied in any particular implementation of the disclosure, and an artisan would recognize from the disclosure herein a myriad of combinations of such aspects, advantages, or features.

### BRIEF DESCRIPTION OF THE DRAWINGS

The foregoing is a summary, and thus, necessarily limited in detail. The above-mentioned aspects, as well as other aspects, features, and advantages of the present technology are described below in connection with various implementations, with reference made to the accompanying drawings.

FIG. 1 illustrates a computing environment including a patient care application for assisting a patient.

FIG. 2 illustrates a flow diagram illustrating one implementation of a permissions architecture for a system for post-health event management.

FIG. 3 illustrates one implementation of a user device for post-health event management.

FIG. 4 illustrates a flow diagram of an overview of a system for post-health event management.

FIG. 5 illustrates an implementation of a graphical user interface (GUI) configured to display data, events, and/or alerts related to the patient.

FIG. 6 illustrates an implementation of a GUI configured to display a task board for individuals that are a part of a care team for a patient.

FIG. 7 illustrates an implementation of a GUI configured to display one or more patient metrics over time or a status of one or more patient metrics.

FIG. 8 illustrates an implementation of a GUI configured to display a status of one or more biometrics and/or an alert progress for one or more individuals.

FIG. 9 illustrates an implementation of a GUI configured to display a recovery status or indicator.

FIG. 10 illustrates an implementation of a GUI configured to prompt a patient for feedback.

FIG. 11 illustrates an implementation of a GUI configured to display a common questions and answers related to the health event.

FIG. 12 illustrates an implementation of a GUI configured to display a plurality of periods of time in which a patient may be monitored or cared for.

FIG. 13 illustrates an implementation of a GUI configured to display one or more periods of time in which a patient may be monitored or cared for.

FIG. 14 illustrates an implementation of a GUI configured to display one or more parameters for a consult between a patient and a healthcare provider.

FIG. 15 illustrates an implementation of a method for customizing the presented data/information according to user preferences and other input parameters.

FIG. 16 illustrates an implementation of a GUI configured to display one or more patient demographics.

FIG. 17A illustrates an implementation of a GUI configured to display a learning lesson dashboard.

FIG. 17B illustrates the GUI shown in FIG. 17A with a notification alert in a learning tab of a ribbon.

FIG. 18A illustrates an implementation of a GUI configured to display an assessment dashboard.

FIG. 18B illustrates the GUI shown in FIG. 18A with a notification alert in an assessment tab of a ribbon.

FIG. 19 illustrates an implementation of a GUI configured to display a question dashboard.

FIG. 20 illustrates an implementation of a GUI configured to display a private conversation board.

FIG. 21 illustrates an implementation of a GUI configured to display a task board for individuals that are a part of a care team for a patient.

FIGS. 22A-22E illustrate an implementation of a mobile application GUI configured to display a note taking process.

FIG. 23 illustrates an implementation of a mobile application GUI configured to display an assessment.

FIG. 24 illustrates an implementation of a mobile application GUI configured to display common questions and answers related to the health event.

FIG. 25 illustrates an implementation of a mobile application GUI configured to display a Navigator Monitoring notification screen.

FIG. 26 illustrates an implementation of a mobile application GUI configured to display a notification alert screen on a mobile device.

FIG. 27 illustrates an implementation of a mobile application GUI configured to display information related to healthcare team members of a patient.

FIG. 28 illustrates an implementation of a mobile application GUI configured to display information related to impairments of a patient.

FIG. 29 illustrates an implementation of a mobile application GUI configured to display a home screen of the mobile application.

FIG. 30 illustrates an implementation of a mobile application GUI configured to display content delivered at a certain time.

FIG. 31 illustrates a flow diagram of an overview of a system for a learning content review process.

FIG. 32 illustrates a flow diagram of an user post-health event system.

The illustrated implementations are merely examples and are not intended to limit the disclosure. The schematics are drawn to illustrate features and concepts and are not necessarily drawn to scale.

### DETAILED DESCRIPTION

### Introduction

The road to recovery following a health event can be challenging and demanding for any party involved. The systems and methods contained herein can offer a path towards recovery in which a user can progress through the stages of recovery. With the assistance of the health application platform, the systems and methods described herein can assist patients and care partners during the recovery process. Following a health event, such as a stroke, patients can be left unsure of their quality of life and may not be aware of the resources available to them. Further, care takers wanting to assist patients may not have the necessary tools or knowledge readily available to effectively assist during the recovery period. Care partners may have to manage hundreds of survivors of a health event. The computing systems and methods described herein can improve management and treatment of survivors. In some implementations, the management and treatment is improved through improvement in user interfaces that addresses challenges in communication with a stroke survivor.

### Overview

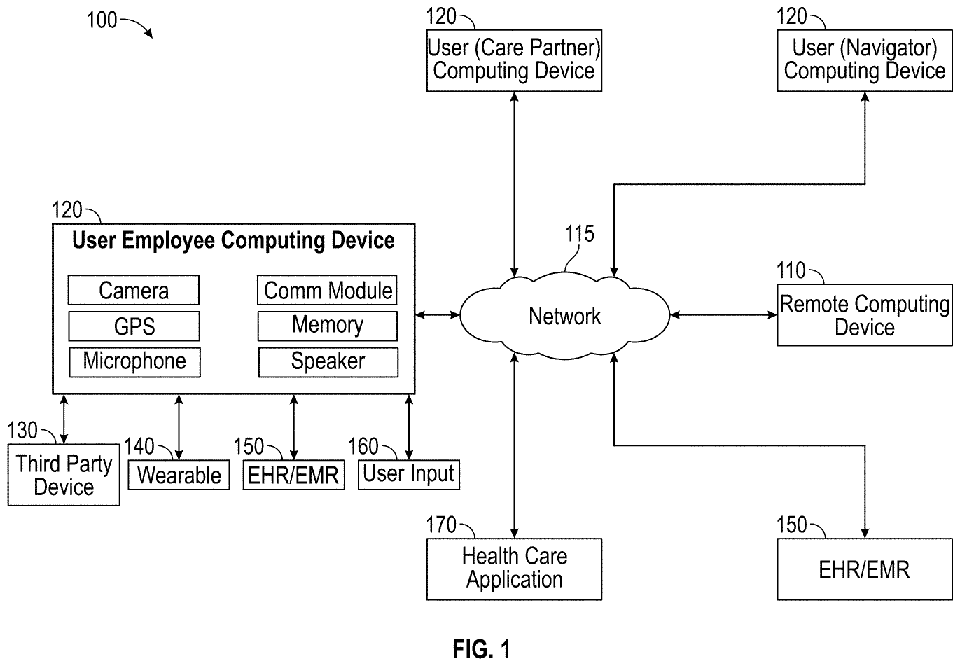

FIG. 1 illustrates a computing environment 100 for post-health event care management. The computing environment 100 can be used in any type of post-health event care such as after experiencing a stroke, for monitoring diabetes after diagnosis, recovering from surgery, for example hip or knee surgery, and any other type of long-term health event where post-supervision can be beneficial. The computing environment 100 can further include a computing devices 120. A user of the computing environment 100, for example, a caregiver, healthcare provider, patient, and/or care team member of the patient may use, interact with, and/or receive information from the user computing device 120. User computing device 120 is illustrated as a single item for convenience but can represent one or more user computing devices 120. In general, the user computing device 120 can include any type of computing device capable of executing one or more applications and/or accessing network resources. For example, the user computing device 120 can be one or more desktops, laptops, netbooks, tablet computers, smartphones, PDAs (personal digital assistant), servers, smartwatches, a combination of the same, or the like. The user computing device 120 can include software and/or hardware for accessing the healthcare system 170, such as a browser or other client software (including an “app”). In some implementations, some or all of the modules of the healthcare system 170 are installed as application software on the computing device 20.

The healthcare system 170 can be implemented in computer hardware and/or software. The healthcare system 170 can execute on one or more remote computing devices 110, such as one or more physical server computers. In implementations where the healthcare system 170 is implemented on multiple servers, these servers can be co-located or can be geographically separate (such as in separate data centers). Additionally or alternatively, the healthcare system 170 can be implemented on one or more virtual machines that execute on a physical server or group of servers. Further, healthcare system 170 can be hosted in a cloud computing environment, such as in Amazon Web Services (AWS) Elastic Computer Cloud or the Microsoft® Windows® Azure Platform.

The user computing device 120 can remotely access some or all of the healthcare system 170 on these servers or through the network 115. The user computing device 120 can include thick or thin client software that can access the healthcare system 170 on the one or more servers through the network 115. The network 115 can be a local area network (LAN), a wide area network (WAN), such as the Internet, combinations of the same, or the like. For example, the network 115 can include any combination of associated computer hardware, switches, etc. (for example, an organization's private intranet, the public Internet, and/or a combination of the same). In some implementations, the user software on the user computing device 120 is a browser software or other application. The user computing device can access the healthcare system 170 through the browser software. In certain implementations, some or all of the healthcare system 170's functionality can be implemented on the user computing device.

The user computing device 120 can receive user input 160 (e.g., via one or more user input elements via a graphical user interface) from a user, for example, biographic data, symptom data (e.g., based on self-reporting, fatigue, mood, pain, diet, spasticity, etc.), emotion data, questions for a healthcare provider, requests for help to caregivers, patient-initiated assessment (e.g., general, lifestyle, home safety, fall risk, etc.), and the like. Further, the user computing device 120 may receive data, software updates, healthcare provider information (e.g., recommendations, answers to questions, etc.) from a remote computing device 110, for example a server or remote workstation.

The user computing device 120 may be communicatively coupled, for example, directly or indirectly, to one or more devices that can include a third-party device 130, a wearable device 140, or an electronic health record or medical record 150 (EHR/EMR) such that the user computing device 120 receives data or information from any one or more of these sources or devices 130, 140, 150. The data or information may include, but not be limited to, activity tracking data (e.g., steps, minutes of activity, etc.), heart rate data, breathing rate (e.g., indicator of stress level), blood pressure (e.g., from a communicatively coupled cuff, manually input, EHR/EMR), blood oxygen saturation, blood sugar (e.g., from a communicatively coupled glucose monitor, manually input, EHR/EMR), and/or clinician-generated assessment data (e.g., PROMIS questionnaires, Fugl Meyer, ARAT, PHQ2/9, etc.) stored in an EHR/EMR or completed directly in the application. Some possible wearables that are configurable with the present systems and methods are described in related International Patent Application PCT/US2020/055604, filed Oct. 14, 2020, the contents of which are herein incorporated by reference in their entirety. Other possible wearables that may be configured to work with the present systems and methods include devices, systems, or wearable available from Apple®, Fitbit®, Garmin®, Samsung®, and/or Beats® or pedometers, blood pressure cuffs, SpO2 monitors, heart rate monitors, and/or scales.

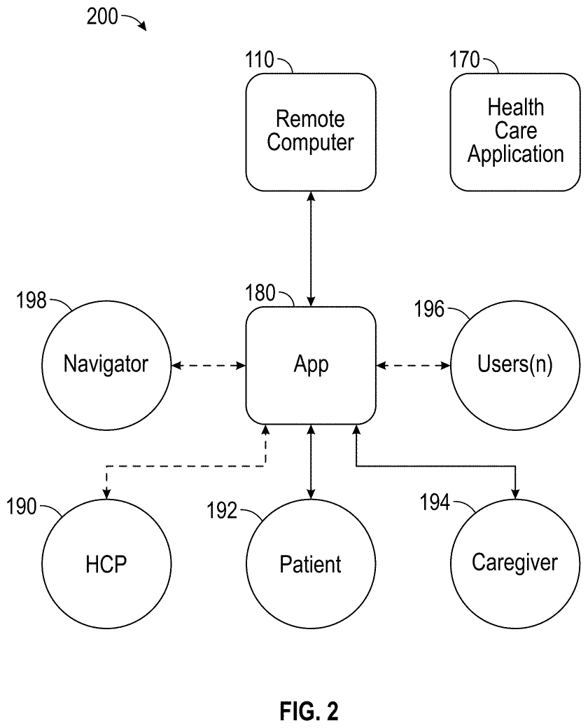

FIG. 2 illustrates a permissions architecture for a system for post-health event management. A system 200 for stroke care management may include various permission layers such that a variety of users may interact with an application 180 on his/her own device but may only have access to needed information and may be denied access to certain information. The healthcare system 170 does not have to reside on a client's device. For example, it could execute on a server or even a monitoring device, if the monitoring device has the necessary computing capabilities. The healthcare system 170 can also execute across multiple devices, such as the client device(s), one or more remote servers, as well as one or more monitoring devices.

The permissions may be set by an administrator, for example a healthcare provider, a primary caregiver, or the patient. The application 180 may be downloaded from a remote computing device 110 onto each user's personal device or a remote workstation, a mobile device, a wearable device, a laptop, desktop, etc. to which the user has access. The application 180 may have various levels of permissions such that the patient 192 has access to all information and resources. A caregiver 194 may have access to an overall view of the patient's health and/or wellbeing, a healthcare provider interface (e.g., to ask questions of or interact with the healthcare provider, etc.), caregiver resources, etc. An optional user, such as a healthcare provider 190, may have access to all the patient's health information (e.g., historical and in real-time), user input at least related to symptoms and emotions, etc. An optional user, such as a navigator 198, may have access to patient onboarding materials and biographic data to help a patient and their care team to begin to use the application and related materials. The system 200 may have any number of users 196 with varying levels of permission or access to system features and components. For example, some users may only receive alerts from the system 200, for example in the event that the patient has a recurrence or has reached a milestone. Some users may only receive task requests from the system 200, for example to help the patient manage day to day activities and tasks, to transport the patient to and from appointments, to provide meals to the patient, to monitor the patient, etc.

Still referring to FIG. 2, a healthcare system 170 (or the healthcare application 80) manages the overall software system. The healthcare system 170 may reside and execute in whole and/or partially on one or many computing devices, including, but not limited to, the remote computing device 110 and the user computing device 120 (see FIG. 1). The healthcare system 170 may provide and/or allow access to different features of the system 170 depending upon the type of user. For example, a user who is a patient may receive features and information or have access privileges that are different than a user who is a medical caregiver or an administrator. The healthcare system 170 may also receive, process, and provide confidential data relative to users of the application. It will be appreciated that the confidential data may not be provided or accessed by all users; rather, the confidential data may be restricted to specific individuals, such as the patient, the medical provider, the insurance provider, and/or the application administrator. Confidential data may be stored on the user computing device 120; additionally, or alternatively, confidential data may be encrypted and stored on the remote computing device 110; or all or a subset of the confidential data may be stored in both systems. Other alternatives include anonymizing the patient-specific data, which may be used in other ways, such as data analytics and machine learning algorithms. The confidential data may be accessed when appropriate credentials, such as username, passwords, token numbers, decryption keys, or combinations and equivalents thereof, are submitted. In this manner, only authorized users with the appropriate credentials are capable of accessing the protected confidential data.

### Patient Care Device

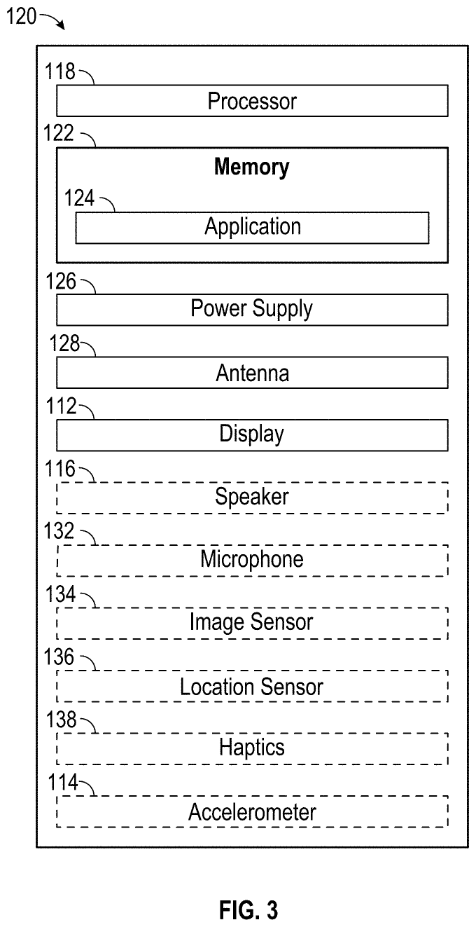

FIG. 3 illustrates a user computing device 120 for post-health event care management. Hardware processor 118 may be coupled, via one or more buses, to memory 122 to read information from and optionally write information to memory 122 (e.g., RAM, ROM, flash memory, EEPROM, a hard disk drive, a solid-state drive, etc.). Any of the software described herein may be programmed into memory 122 or downloaded as an application 124 onto memory 122 and executed by hardware processor 118. In some implementations, the memory 122 is configured to store one or more metrics of the user from the user's interaction with the user device 120. Hardware processor 118 may comprise a general-purpose processor, an application specific integration circuit (ASIC), a field-programmable gate array, a dual core processor, a single core processor, a microprocessor, a digital signal processor (DSP), an embedded processor, any programmable logic device, or any combination thereof. In some implementations, one or more hardware processors 118 are further configured to generate analysis based on the stored one or more metrics. The one or more hardware processors 118 can further be configured to change a timing of delivery of learning content based at least on the generated content.

In some implementations, a user computing device 120 further includes a power supply 126. Power supply 126 may include a rechargeable battery (e.g., Lithium-ion battery), a disposable battery, solar energy-based source, kinetic energy-based source, or other renewable energy source. Power supply 126 may provide energy for one or more components in user computing device 120, for example one or more sensors, hardware processor 118, memory 122, etc.

In some implementations, user computing device 120 includes an antenna 128 communicatively coupled to the hardware processor 118. Antenna 128 may receive and demodulate data over a communication network and/or prepares and transmits data over a communication network. Antenna 128 may act as a receiver, transmitter, or both (i.e., transceiver). Alternatively, or in addition to antenna 128, a data bus (e.g., serial or parallel) may be included to receive data from, or send data to, one or more sensors from memory and/or hardware processor via a wired connection.

In some implementations, user computing device 120 includes a display 112 communicatively coupled to the hardware processor 118. Display 112 may present one or more GUIs based on user input; inputs from one or more devices, as shown in FIG. 1; based on user selection of various elements or indicators presented in a GUI; etc. The display 112 may include a visual display with or without touch responsive capabilities (e.g., Thin Film Transistor liquid crystal display (LCD), in-place switching LCD, resistive touchscreen LCD, capacitive touchscreen LCD, organic light emitting diode (LED), Active-Matrix organic LED (AMOLED), Super AMOLED, Retina display, Haptic/Tactile touchscreen, or Gorilla Glass). A GUI of the display 112 may be updated based on one or more user inputs. For example, content presented on the GUI may be moved to a different portion of the GUI; enlarged or reduced in size; presented more audibly, textually, or haptically; presented in a more basic or complex manner; etc. at least based on a symptom or disability of the patient, as described elsewhere herein.

In some implementations, content presented to a user includes a set of curated, personalized learnings based on the patient's specific health event details available at discharge, along with a more expanded (and, in certain implementations, less personalized) library of learning materials and resources that patients can access later on. Some of the information may be linked to or may prompt the user to execute certain actions, for example an article about the importance of medication management may then prompt the user to set up a medication list for tracking over time. Various learning materials may also include understanding one or more complications at the hospital (e.g., aspiration pneumonia, brain swelling, difficulty swallowing, elevated pressure, reperfusion Hemorrhage, salt imbalance, vessel spasm, etc.).

In some implementations, user computing device 120 optionally includes speaker 116 and/or microphone 132. Such components may provide greater accessibility to systems for post-health event care management. For example, depending on a disability of the patient, a GUI presented on display 112 may be updated to enhance accessibility for the user. In one embodiment, a patient that experienced a stroke in the brainstem region may suffer from total or partial alterations in hearing or vision or a patient suffering from MS may experience vision loss. As such, a GUI presented on display 112 may be updated at any time so that the user may interact with the GUI primarily through audible means (i.e., utilizing speaker 116 and/or microphone 132) in situations of vision loss or through visual means (i.e., relying more on text or tactile based inputs and outputs) in situations of hearing loss. In another embodiment, a patient that experienced a stroke in the cerebral cortex or a patient suffering from amyotrophic lateral sclerosis may have difficulty with verbal expression, auditory comprehension, or may present with dysarthria. As such, a GUI presented on display 112 may be updated to present information using text or tactile based inputs and outputs. In still another embodiment, a patient that experienced a stroke in the central nervous system (e.g., spinothalamic tract, corticospinal tract, dorsal column) may suffer from hemiplegia such that tactile interaction with the GUI may be difficult. As such, the GUI presented by display 112 may be updated such that the user primarily interacts with the GUI via audible (e.g., using microphone 132, speaker 116, etc.) and/or visual means. Stroke location and/or type, symptoms, disabilities, etc. may be received from an EHR/EMR, wearable, remote computing device, etc. such that a hardware processor of the system receives these inputs, determines how information should be displayed/output to the user based on the received information (and/or any other information in the system), and updates a GUI presented by the display based on said determining.

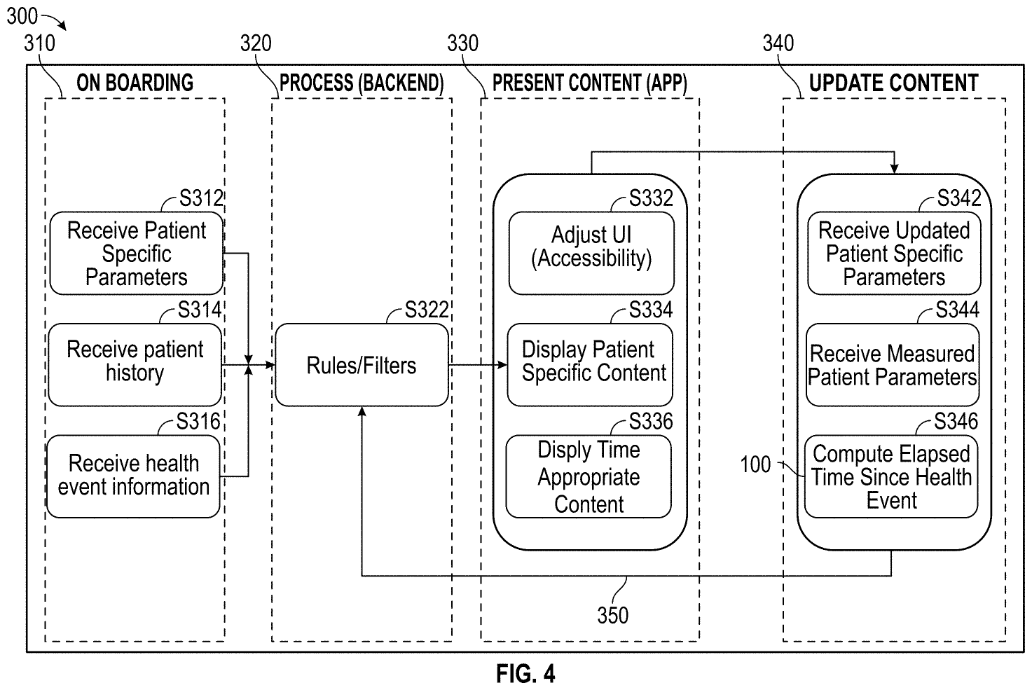

In some implementations, speaker 116 and/or microphone 132 is further, or alternatively, used to determine a speech quality of the user. For example, a speaker 116 may prompt the user to speak and/or a user may speak into the microphone, such that a speech of the user may be analyzed by the processor, and an indication of speech quality may be output. For example, a speech quality indicator may be based on, but not be limited to, slurred speech, disorganized speech, dysarthria, etc. A quality of speech over time of the patient may be input into the system or received by the system to assess a recovery or health quality of the patient over time, as shown in FIG. 4, to better tailor user care.

In some implementations, user computing device 120 optionally includes an image sensor 134. Image sensor 134 may be used to image a body portion of the user, for example a face to detect one or more emotions, facial symptoms (e.g., eyelid drooping, facial muscle weakness, etc.); one or more limbs to detect, for example muscle spasticity, flaccidity, a gait or balance of the patient, etc.; etc. Such information may be input or received by the system to assess a recover or health quality of the patient over time, as shown in FIG. 4, to better tailor user care.

In some implementations, user computing device 120 optionally includes a location sensor 136, for example a global positioning device. Location sensor 136 may be configured to determine a location of a patient in the patient's home, for example to monitor a patient when he/she is alone; to monitor whether the patient is immobile during periods of time in which the patient is typically mobile (possible indicating a health event like falling or stroke); to monitor a patient when he/she is away from home; etc. Location sensor 136 may be used independently or together with accelerometer 114 to determine whether the patient is exhibiting normal activity or activity that may be indicative of a health event. In some implementations, location sensor 136 additionally, or alternatively, tracks a frequency with which a patient leaves her home, how frequently she is stopping during her route, how long she is away from her home, how far (distance) does she go from her home, elevation changes, how many places does she go, etc. as leading or lagging indicators of her wellbeing, stress, depression, and/or the like.

In some implementations, accelerometer 114 is further used to assess one or more symptoms, disabilities, or a recovery status of the user. For example, accelerometer may be used to determine a gait, balance, muscle weakness, motor activity quality, muscle spasticity, muscle flaccidity, vertigo, coordination, activity, etc. of the user. These accelerometer data may also, or alternatively, be used as leading or lagging indicators of her wellbeing, stress, depression, etc.

In some implementations, user computing device 120 optionally includes haptics 138, for example piezo electronics, eccentric rotating mass motors, linear resonant actuators, etc. In some implementations, a GUI presented by display 112 is updated or personalized to provide haptic feedback provided by haptics 138. For example, in instances where user has visual and/or auditory impairments, information may be communicated to the user, at least in part, via haptic feedback.

The healthcare system 170 can optionally consider multiple factors in determining how to present data and/or information to the users. More specifically, if the system, which includes the user computing device 120 and the system 170, determines that there is an elevated probability of at least one sensory impairment, the system 170 may be configured to modify the output format of data/information accordingly. In some implementations, the system 170 determines the elevated probability of at least one sensory impairment from an input by a user, caregiver, medical records, and the like where data may be retrieved from. User preferences may also be used to determine how to present data/information to users. For example, a user may prefer a certain type or style or font of visual presentation. User preferences, such as the visual presentation, are considered by the healthcare system 170, and these preferences may be modified over time by either or both the user and the system 170. Another consideration that may alter how data/information is presented to users is the computing device or devices they are utilizing. For example, different devices may have different displays, and the type and size of the particular display is an important factor in how data/information is presented to users.

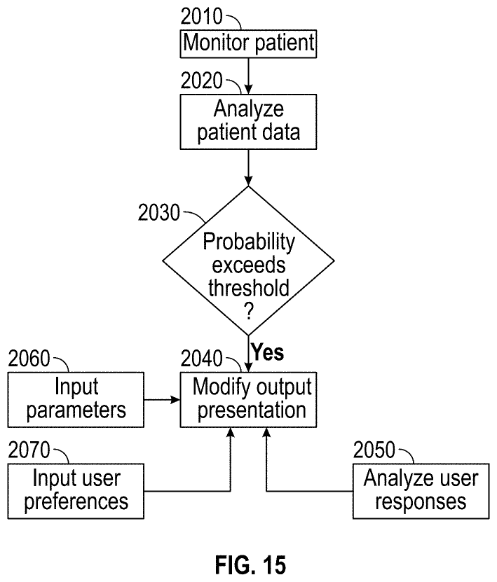

FIG. 15 shows an implementation of a method for customizing presented data/information according to user preferences and other input parameters. In 52010, the healthcare system 170 and associated devices are configured to monitor, track, compile, and store data associated with a user or patient. The monitoring devices comprise one or more of: human-made devices, such as wearables, sensors, computers, etc., a human being, or service animal. The compiled data associated with biometrics, characteristics, and/or parameters of the monitored patient include, but are not limited to, sensory characteristics and measurements, such as temperature, heart rate, gait, vision, hearing, and speech, as discussed herein. It will be appreciated that monitoring the patient may provide invaluable data over time in order to determine if the patient is medically impaired, such as, for example, a hearing or vision impairment. In 52020, the healthcare system 170 receives and/or retrieves the monitored data from the user computing device 120. It will be appreciated that the user computing device 120 may automatically transmit, or push, the monitored data at regularly scheduled times or during a user event, as well as, additionally or alternatively, transmit the monitored data when a request, or pull, command is received from one or more of: the user, the medical practitioner, the caregiver, and/or the healthcare system 170.

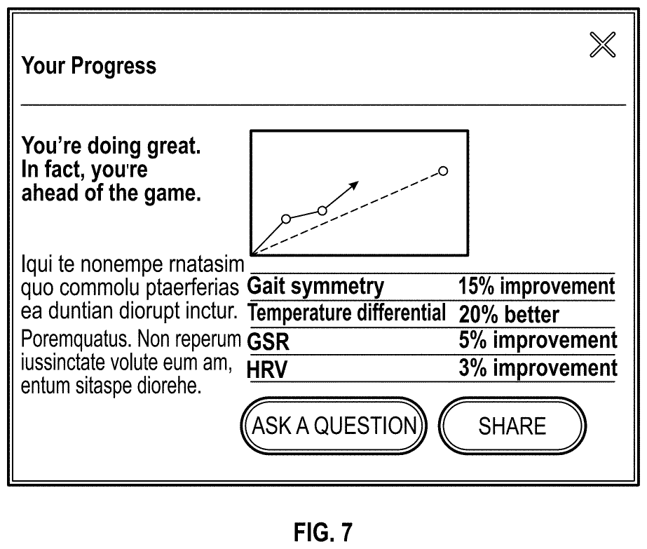

The healthcare system 170 analyzes and processes the monitored data in S2020. Analyzing and processing may involve machine learning systems including, but not limited to, classification, regression, and/or clustering techniques, linear algorithms; regularization techniques, such as lasso, ridge, and elastic net; approaches such as random forest, decision trees, nearest neighbors, support vector machines, gradient boosting, neural networks, deep learning techniques, etc. The monitored data may be provided in various formats, including, but not limited to: numerical, text, natural language, video, image, and other formats, and may also include both structured and unstructured data. The healthcare system 170 receives and processes the monitored data regardless of the different formats. The healthcare system 170 analyzes the monitored data in order to determine the probability that the patient experienced, or is currently exhibiting, any impairment. More specifically, the analysis assesses the data using one or more, but not limited to, the following: machine learning algorithms, various sensory baselines, or trends of recorded data associated with the patient. Based on the analysis, the healthcare system 170 is configured to assign a probability of impairment, such as a visual, hearing, and/or a speech impairment. It will be appreciated that the analysis may use patient-specific data, such as their baselines, trends, and previously received data, such as shown in FIG. 7. Additionally, the analysis may also use a trained machine learning system and algorithm based on anonymous patient data detailing various impairments across groups of users. Further, the healthcare system 170 may develop several algorithms based on anonymous patient data that can be grouped by gender, age, race, and/or health event. The probability is then compared to a predetermined threshold in S2030, and, in the event the probability exceeds the predetermined threshold, the output presentation of data/information is modified in S2040 according to predefined specifications.

Several output presentation modifications may be defined in the event the probability exceeds the predetermined threshold. It will be appreciated that modifications to the output presentation may occur at any time over the monitoring period. For example, in response to an elevated probability of a visual impairment, the healthcare system 170 may modify the output presentation of visual data/information text and/or graphics by one or more of the following: provide corresponding or enhanced auditory output to the patient; modify the visual output presentation, such as increasing a font size of the text or changing the brightness of the text and graphics. As another example, in response to an elevated probability of a hearing impairment, the healthcare system 170 may modify the output presentation of auditory data/information by one or more of the following: provide corresponding visual texts and graphics; increase a volume level of transmitted auditory sounds provided to the patient; modify the frequency level to a higher or lower frequency; or transmit a command to the user computing and/or monitoring devices to raise the volume of the speaker. Further, in response to an elevated probability of a speech impediment, which would reduce an accuracy of a speech recognition interface provided by the healthcare system 170, the healthcare system 170 may modify the output presentation by one or more the following: solicit input responses from the patient via text, such as through a keyboard or other user-selectable display options, such as response buttons or a menu; presenting a text chat box. In further examples, in response to an elevated probability of visual, auditory, speech, and/or other impairment in the patient, the healthcare system 170 may also automatically contact another human being, such as a healthcare provider or emergency contact.

Additional factors that may also prompt modifications in the output data/information provided to users can include, for example, the type of computing device or coupled monitoring device that the user is using or initialized. More specifically, in 52060, the healthcare system 170 receives input parameters regarding one or more computing devices. Input parameters could include computing device type and capabilities, coupled monitoring device types, such as display, camera, microphone, speaker, sensors, etc., memory size, data output format, transmission protocols, etc. The healthcare system 170 receives the input parameters at block 52040 and may modify the data/information output to correspond with the parameters of the device(s) being used. For example, the healthcare system 170 may be configured to increase font sizes for devices having a larger display or provide auditory sounds for devices having a speaker.

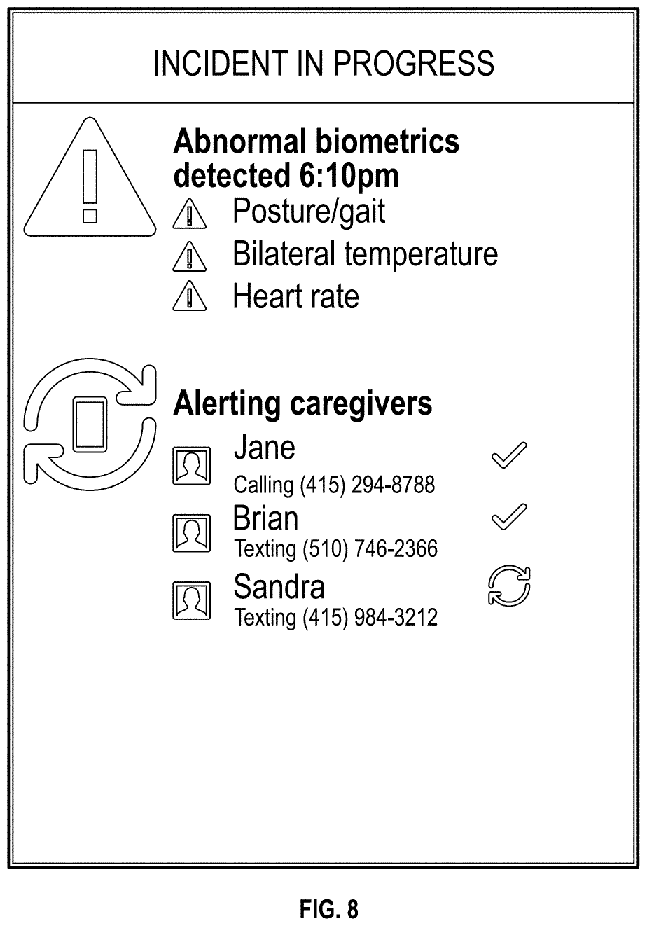

The healthcare system 170 may also be configured to receive user preferences regarding the type or format of desired data/information output presentation. For example, the output presentation may be configured to provide an enhanced sound output or an enhanced visual output, or both, depending on user preferences. Further, the output presentation may be configured with varying output presentations based on the time of day. For example, a user preference may specify sound outputs during periods when they are away from the display and visual outputs during periods when they do not want to be disturbed by sound. User preferences may also include muting some or all of the output presentations during periods in which users may be sleeping. In some situations, the healthcare system 170 detects an emergency when a patient may be sleeping, for example, and may be configured to output an alarm, other loud sound, and/or vibration in order to wake them. User preferences may also be configured to store emergency names and numbers (see FIG. 8), such as a healthcare professional, hospital, caregiver, and/or family member, so that they may be contacted for any predetermined in-progress or previous event, such as a device-detected fall, potential extreme impairment, such as a possible stroke, a device-triggered alarm, and/or a nonresponsive user.

The healthcare system 170 may also monitor the responses users provide in order to customize the type of output. In S2050, the healthcare system 170 is configured to analyze user responses over time and modify the output presentation accordingly in order to provide output presentations that are most meaningful for every user. For example, the healthcare system 170 may provide output presentations in different visual styles, such as, different font sizes, colors, and/or placement of information. The healthcare system 170 is configured to determine the visual style that results in the most responsiveness from the user and may modify or vary the output format to optimize the presentation and responsiveness from the user. As another example, the healthcare system 170 may provide output presentations using both sound and visual information. Depending on the responsiveness from the user, the healthcare system 170 may provide an emphasis of one or both sound and visual information. For example, the healthcare system 170 may determine that a user is more responsive to provided visual information, and the output presentation for that user is modified or updated for an emphasis on visual information. Alternatively, the healthcare system 170 may determine that the user is more responsive when both visual and auditory output information is provided, and the output presentation for that user is modified or updated to provide both visual and auditory information.

In some implementations, the hardware processor, for example on a local or remote computing device 110, is configured to output information to a user, for example a care team, a patient and/or a caregiver. (see FIG. 1). The information that may be outputted includes, but is not limited to, signs of a health event (e.g., stroke), health event prevention (e.g., stroke), underlying health issues education or information, information related to the type of health event the patient experienced, financial resources or impact information, what to expect after experiencing a health event, returning to work resources, diet and/or nutrition education, smoking cessation materials or programs or actions, physical activity and/or exercise resources, behavioral resources, general vocabulary and definitions so the patient can get the most out of appointments and/or resources, common questions for your healthcare provider, etc.

In some implementations, the software further includes one or more tools for a user, for example a patient or caregiver. The tools may help manage disabilities of a user, for example a bathroom finder may be included to help manage incontinence; games may be included to help improve memory; exercises or games to help the patient develop new ways of functioning/understanding/conversing/etc. The tools may help manage underlying health issues, for example high blood pressure, heart conditions, chronic kidney disease

### Methods for Personalization of Post-Health Event Care Management

FIG. 4 shows a flow diagram of an overview of a system 300 for post-health event care management such as, for instance, curating content for a user. System 300 can be implemented by any one of the systems mentioned herein. For illustrative purposes, the system 300 will be described as being implemented by components of computing environment 100 of FIG. 1. The system 300 depicts an example overview of curating learnings. In some implementations, the user is presented curated content which can include a set of curated, personalized learnings at least based on the patient's specific health event details available at discharge, along with a more expanded (and, in certain implementations, less personalized) library of learning materials and resources that patients can access later on. Some of the information may be linked to or may prompt the user to execute certain actions, for example an article about the importance of medication management may then prompt the user to set up a medication list for tracking over time. Various learning materials may also include understanding one or more complications at the hospital (e.g., aspiration pneumonia, brain swelling, difficulty swallowing, elevated pressure, reperfusion Hemorrhage, salt imbalance, vessel spasm, etc.

In general, the system 300 may include various stages, for example an onboarding stage 310; a processing stage 320; a content presentation stage 330; and an updated content stage 340. Stages 320, 330, 340 may be repeated any number of times, as shown by arrow 350 and/or various inputs at blocks S342, S344, S346. The various inputs at blocks S342, S344, S346 can be fed back into the rules and/or filters at block S322 to further adjust the GUI, update the content, and/or achieve timely delivery of content. For example, stages 320, 330, 340 can be repeated every time a new or updated input (e.g., user input, input from EHR/EMR, input from wearable, input from third-party device, etc.) is received; automatically based on a certain time interval or various criteria; manually based on user request or user selection; or based on any other criteria or parameters.

Any process descriptions, elements, or blocks in the flow diagrams described herein and/or depicted in the attached figures should be understood as potentially representing modules, segments, or portions of code which include one or more executable instructions for implementing specific logical functions or steps in the process. Alternate implementations are included within the scope of the implementations described herein in which elements or functions may be deleted, executed out of order from that shown or discussed, including substantially concurrently or in reverse order, depending on the functionality involved, as would be understood by those of ordinary skill in the art.

Turning now to onboarding stage 310, which includes one or more of: receiving one or more confidential patient specific parameters at block S312; receiving a history of a patient at block S314; and/or receiving information about one or more health events of the patient at block S316. For example, patient specific parameters may include, but not be limited to: name, date of birth, contact information (e.g., phone number, email address, etc.), biographic data, demographics (e.g., living situation, financial situation, etc.), identity, goals, and the like. Further for example, history of a patient may include, but not be limited to: underlying health conditions (e.g., high blood pressure, diabetes, etc.), social history, medical history, surgical history, family history, tests or assessments, clinical data, etc. Still further for example, health event information may include, but not be limited to: a type of health event (e.g., type of stroke, type of multiple sclerosis, etc.), a frequency of the health event (e.g., frequency of relapses, frequency of symptoms, etc.), a date of the health event, a location of treatment, a location of the health event, a location of the health event within the body (e.g., location of stroke within the brain, location of demyelination in multiple sclerosis, etc.), one or more disabilities as an outcome of the health event, one or more outcomes as a result of the health event, and the like. Information for any of blocks S312, S314, S316 may be received by the system via user input, pulled from one or more databases (e.g., EHR/EMR, profile on third party platform, etc.), or otherwise pulled form one or more devices (e.g., wearable, third party device, remote computing device, etc.).

One or more patient specific parameters may be displayable over time, for example to determine a trend or trajectory for the user. Symptom trends may be invaluable for a user to determine his/her progress.

In some implementations, in response to received symptom data, the application provides resources, a list of one or more future symptoms that may occur, future signs or symptoms to watch for, and/or on-going concerns as a result of the symptom. Table 1 below shows various symptoms and possible outcomes, results, or additional issues/symptoms for which a care team or the application can monitor.

TABLE 1

| | Related Symptoms or Results or |
|---|---|
| Symptom | Causes for Concern |
| Aphasia | Interacting with strangers |
| Apraxia of Speech | Interacting with strangers |
| Fatigue | Triggers and/or patterns |
| Hemiplegia on left or right side | Pressure ulcers |
| | Shoulder subluxation |
| | Fall risk |
| | Edema |
| Impaired balance | Fall risk |
| Impaired vision | Fall risk |
| | Spatial awareness |
| Neglect on right or left side | Fall risk |
| | Spatial awareness |
| Personality changes | Triggers and/or patterns |
| Seizures | Triggers and/or patterns |
| Short-term memory loss | Watching for medication mistakes |
| Difficulty paying attention | Watching for medication mistakes |
| Dysphagia | Oral hygiene |
| | Aspiration Pneumonia |
| | Choking |
| | Signs of dehydration |
| | Signs of malnutrition |
| Incontinence | Signs of urinary tract infection |
| | Signs of dehydration |
| | Signs of constipation |
| Sensory disorders | |
| Hydrocephalus | Signs of shunt complications |

In some implementations, as shown at block S322 of stage 320, the one or more inputs from blocks S312, S314, S316 are processed. For example, processing at block S322 may include using various rules and/or filters to determine one or more resources (e.g., videos, support groups, written materials, appointments, etc.) that the patient needs to support his/her recovery; determine a likelihood of the patient developing one or more symptoms or disabilities; determine a likelihood of partial or full recovery; determine a treatment and/or therapy plan (e.g., pharmaceuticals, physical therapy, appointments, surgeries, etc.) for the patient; determine a care plan (e.g., in conjunction with one or more caregivers) for the patient; determine a safety plan (e.g., recommended home safety adjustments, recommended work safety adjustments, recommended mobility adjustments, etc.) for the patient; etc.

In some implementations, the software includes tools, action items, and/or information for creating a care plan for the patient. The care plan may include clinical action plans or non-clinical action plans. The care plan may be personalized for a patient and/or dynamically updated over time based on one or more user inputs or inputs about a user. The care plan may be focused on the first few weeks after a health event, after the initial few weeks, or for any length of time or time frame. In some implementations, a care plan includes a rehabilitation plan, for example that includes personalized exercises for the patient based on the patient's health event type and/or disability. Optionally, the care plan may include non-clinical action plans, for example, but not limited to, home improvements to increase the safety of the patient in their home (e.g., fall prevention) and/or paperwork that the patient should have on file (e.g., advanced directive, power of attorney, etc.). Optionally, the care plan may further include clinical action plans such as smoking cessation programs and/or blood pressure lowering plans. Also, the care plan may include tools to facilitate the user in navigating the healthcare system.

In some implementations, as shown at stage 330, a graphical user interface presented on a display of a user device is updated to improve accessibility of the display for the user at block S332, display patient specific content at block S334, and/or to display time appropriate content at block S336. For example, and as described elsewhere herein, the type of health event may dictate the type or types of symptoms and/or disabilities that the patient will experience at any particular point in time. Patients may experience muscle weakness or loss of function, vision changes or loss, hearing changes or loss, mental capacity changes, etc. As such, a GUI of a user device may update to accommodate these changes in abilities, symptoms, and/or disabilities so that the user can easily and effectively interact with the system. This may include switching to audio content delivery, text delivery, haptic delivery, switching a distribution or location of material on the GUI, changing a complexity of the material presented, etc. or a combination thereof. Additionally or alternatively, the GUI may update to divide and/or separate content into different distinct pages based on the user's impairment. For example, at least based on the impairment, the font, images, selectable elements, and the like can be adjusted causing the content to be divided amongst divided section and/or pages. Further, the user can input preferences to adjust GUI parameters to further adjust the presentation of the content. In some implementations, the symptom or disability may impact the patient asymmetrically, such that the GUI is updated to accommodate this asymmetry. For example, when a patient experiences vision loss or motor function loss on a left side, content and/or inputs may be positioned toward a right side of the GUI. Additionally or alternatively, a GUI may be updated to have enhanced speech recognition capabilities, such as when a patient is experiencing altered speech or is having difficulty articulating well. Further, a GUI may be updated to increase or reduce complexity of the content that is delivered based on a perceived cognitive capacity of the user, for example severely mentally impaired patients, as a result of their health event, may receive more basic content while patients that are more severely impaired in motor function, but not cognitively, may receive more complex content.

In some implementations, as shown at block S334, content in one or more databases is filtered and presented on a GUI to the user based on patient specific information. For example, the content may be filtered based on a type of health event; type of symptoms and/or disabilities currently being experienced by the patient or expected to be experienced by the patient at a future time point; type of treatment or therapy prescribed for the patient; based on patient reported symptoms, emotions, etc.; based on a history of a patient; based on a support network of the patient; etc.

In some implementations, as shown at block S336, content is selected and/or displayed in a GUI to the user based on time appropriateness. For example, time appropriateness may be based on at least an amount of elapsed time from the health event; a projected health trajectory based on a severity of the health event; a prescribed treatment or therapy; a history of the patient; and/or symptoms and/or disabilities experienced by the patient.

Turning to stage 340, which includes one or more of: receiving updated patient specific parameters at block S342, receiving measured patient parameters at block S344, and/or computing elapsed time since a health event at block S346. Patient specific parameters, as described in connection with block S312, may be updated over time. For example, they may be updated in response to a request or prompt to verify and/or update one or more parameters; updated at a predefined interval; updated when there is a change in any one or more of the parameters; etc. Further, additional inputs may be received by the system over time, for example, one or more measured parameters may be received by the system. For example, a step rate, blood pressure, blood oxygen saturation level, weight, activity level, heart rate, heart rate variability, etc. may be collected by one or more wearables, third party devices, etc. communicatively coupled to the system. The system may then update a GUI presented on the display, content displayed to the patient, and/or timing of content delivery. The system, at block S346, may further be configured to compute an elapsed time since the health event. Such calculated timing may be used to further filter content so that the displayed content is specific for the patient for where they are in their recovery and/or based on a current status of their symptoms and/or disabilities post the health event.

In some implementations, one or more patient specific parameters includes completing a clinical self-assessment, for example a Taking Charge after Stroke (TaCAS) assessment, a Mood Assessment (Patient Health Questionnaire-2, Mental Component Summary of the Short Form 36); an Autonomy-Mastery-Purpose-Connectedness (AMP-C) Assessment; an Activation Assessment (Patient Activation Measure); a medication adherence assessment (Medication Adherence Questionnaire), Modified Rankin Scale for Neurologic Disability (mRS; measures the degree of disability or dependence in the daily activities of people who have suffered a stroke or other causes of neurological disability), patient health questionnaires (PHQ), PROMIS GH (Patient-Reported Outcomes Measurement Information System Global Health) Scale, or the like. For example, the application may comprise one or more of: image upload or capture capabilities, audio recording (and optionally storing) capabilities, drawing capabilities (e.g., user can draw in the app with using touch responsive capabilities of the app), and/or optional or mandatory questionnaires.

In some implementations, an application for post-health even management also includes one or more questionnaires or assessments to determine a preparedness level of one or more caregivers and/or a strain or stress level of a caregiver (e.g., Modified Caregiver Strain Index).

### Client-Patient Graphical User Interfaces

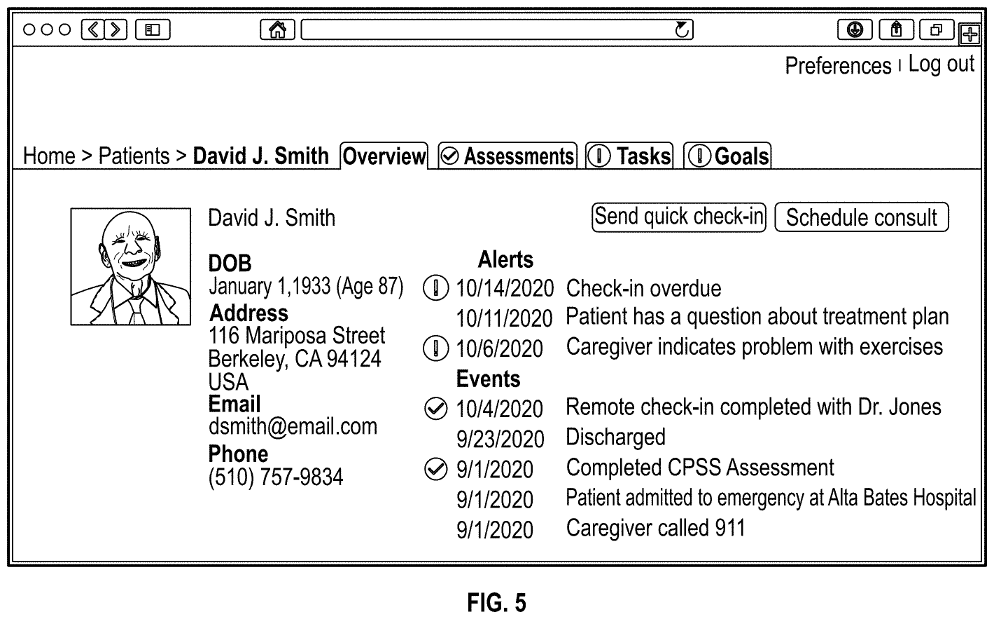

FIG. 5 illustrates an implementation of a GUI configured to display data, events, and/or alerts related to the patient. As described elsewhere herein, FIG. 5 shows various biographic data for the user, in addition to one or more events or alerts for the patient or a user of the system. For example, alerts may be related to check-ins with one or more caregivers, a care team, a physician, etc.; treatment or therapy plan questions or comments; feedback from a caregiver, care team, patient, physician, etc. (e.g., related to exercises, medications, etc.); etc. Events may include appointments (e.g., in-person or remote); assessments; health events (e.g., emergency room visits, readmittances, relapses, etc.); caregiver communications (e.g., messages sent to a healthcare provider, emergency phone calls, etc.).

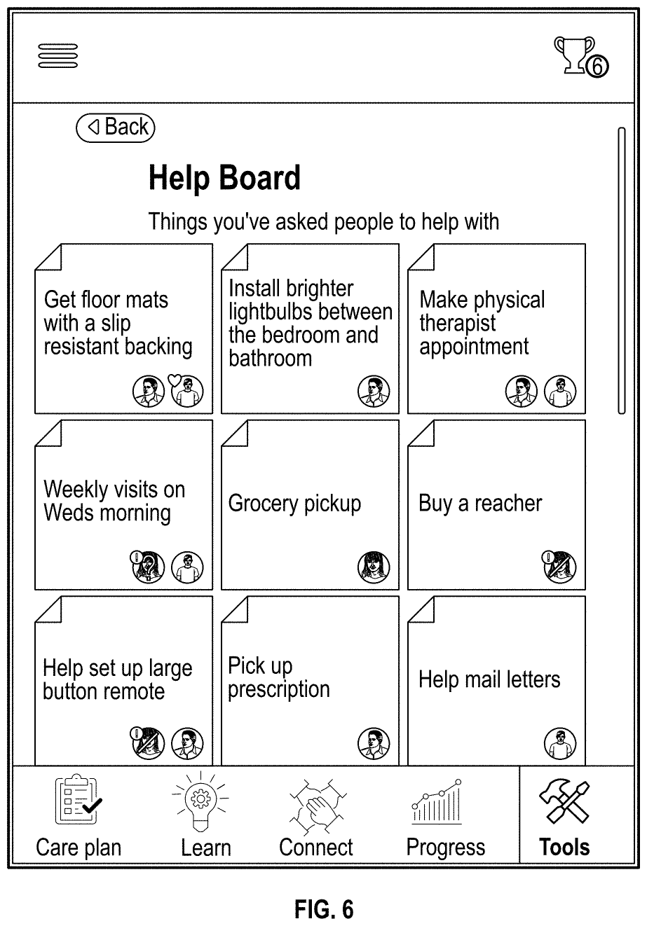

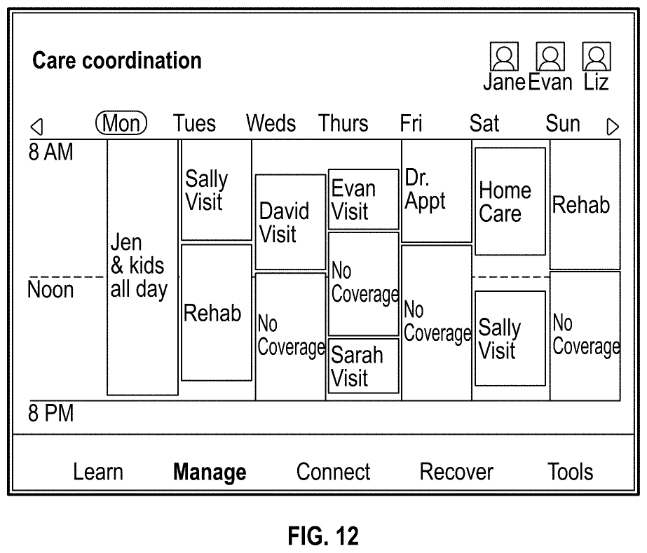

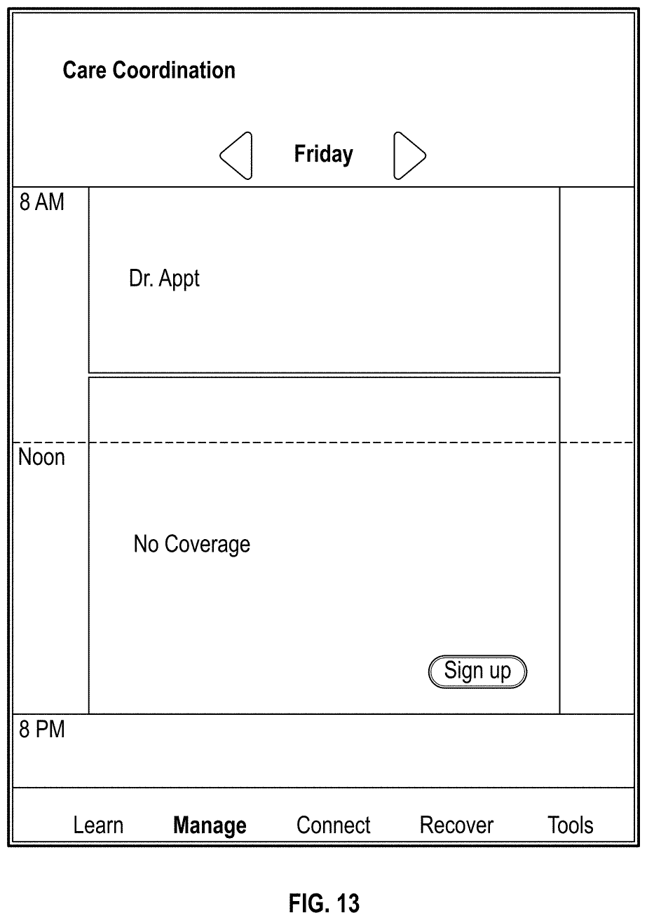

FIG. 6 illustrates an implementation of a GUI configured to display a task board for individuals that are a part of a care team for a patient. For example, a patient or user of the system may assign one or more tasks to various other users of the system. Such tasks may be a part of a care plan for the patient; may be recommended based on a determined safety plan for the patient; may be recommended based on an event or appointment schedule for the patient; and/or may be based on care coordination (e.g., who will be with the patient when). The tasks may be presented as tiles, in a list, linked to and/or in a calendar (shown in FIGS. 12-13). that update position, color, opacity, size, or other indicator based on a status of the task (e.g., outstanding, completed, in progress, etc.). Care coordination may also reveal where there are overlaps or the absence of care for particular time periods, such that various caregivers can elect to sign up for times to care for the patient. In some implementations, a user searches the calendar for one or more sponsored or organized events in his/her community or related to one or more social groups (e.g., in the application, based on social media sites, based on invitations received, etc.). The calendar function may further enable scheduling and/or reminders for appointments, therapy sessions, and/or medications.

FIG. 7 illustrates an implementation of a GUI configured to display one or more patient metrics over time or a status of one or more patient metrics. For example, in some implementations, a GUI presented by a display is updated to show a progress of the patient over time and/or a recovery of the patient over time. The GUI may show various metrics in a tabular format or graphic format. Additionally or alternatively, the GUI may show various metrics as a change over time (e.g., percent, unit of measure, etc.). The GUI may further recommend areas for improvement and/or suggest treatments or therapies to improve various metrics. Selection of input elements on the GUI may enable the user to share the metrics with another user and/or to ask a question of another user, for example a healthcare provider.

FIG. 8 illustrates an implementation of a GUI configured to display a status of one or more biometrics and/or an alert progress for one or more individuals. As shown in FIG. 8, a GUI may be updated to display a potential health event in progress. For example, the GUI may display one or more abnormal biometrics received from a third-party device and/or a wearable. The system may then be configured to transmit a notification or alert to one or more caregivers, a user, healthcare provider, or emergency services.

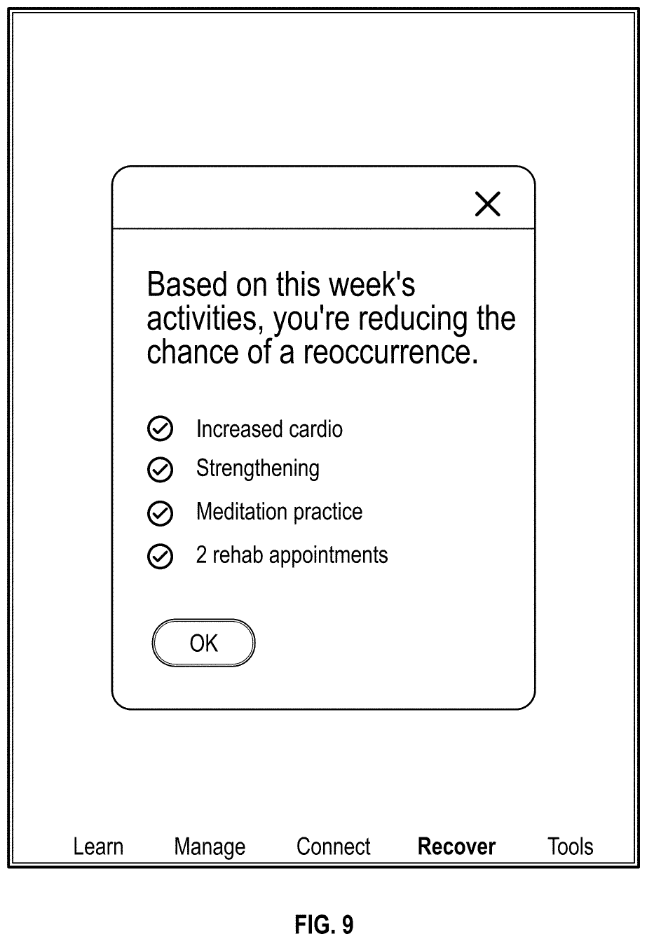

FIG. 9 illustrates an implementation of a GUI configured to display a recovery status or indicator. For example, as shown in FIG. 9, a GUI may be updated to encourage a patient to keep performing certain activities, take certain medications, and/or execute certain therapy regimens to reduce a recurrence or relapse of the health event. Such encouragement may be based on received inputs, for example from patient input data, sensor data from third party or wearables, and/or EHR/EMR data.

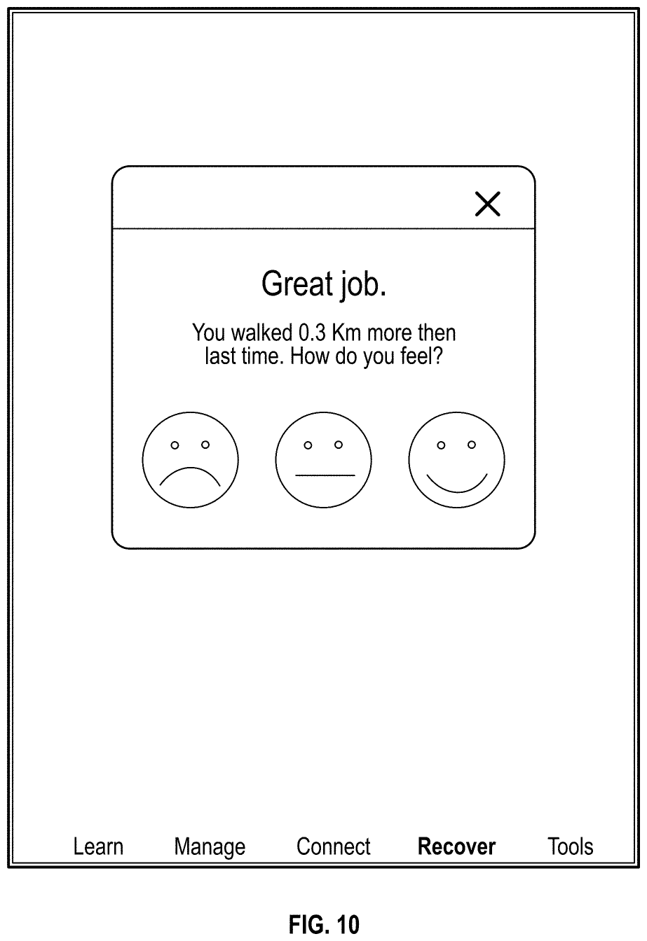

FIG. 10 illustrates an implementation of a GUI configured to prompt a patient for feedback. A GUI may be updated to prompt a user for feedback. For example, a GUI may be updated in response to receiving sensor signals indicative of physical activity, user input about emotions, received assessment data, received caregiver feedback or observations, etc. A patient can also submit their feedback in the form of a picture. The feedback may be related to emotions, overall physical wellbeing, level of fatigue, and/or cognitive wellbeing.

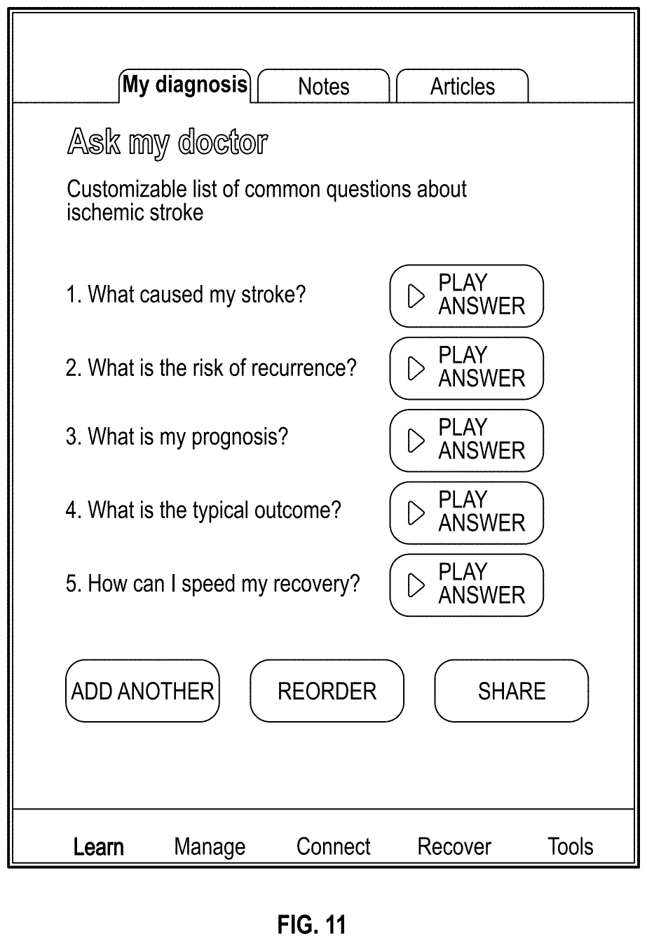

FIG. 11 illustrates an implementation of a GUI configured to display common questions and answers related to a type of health event. In some implementations, curated or personalized content for the patient, for example related to stage 330 in FIG. 4, is presented to the patient in a question-and-answer format, using text, audio files, visuals, etc. based on symptoms or disabilities of the patients.

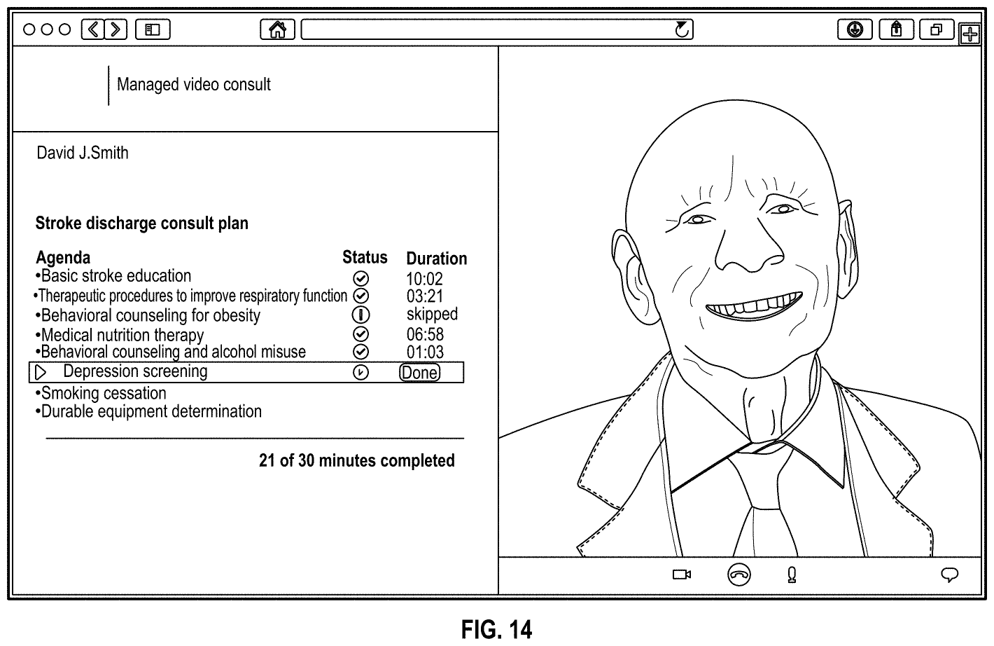

FIG. 14 illustrates an implementation of a GUI configured to display one or more parameters for a consult between a patient and a healthcare provider. In some implementations, the system presented herein is used to perform a consult between a patient or caregiver and a healthcare provider. For example, time spent on various aspects of the visit may be documented by the system; assessments performed; and/or questions asked/answered may be tracked for transmission to a health insurance provider and/or uploaded to an EHR/EMR. Further, the GUI may be configured to display topics and/or questions for the clinician to follow during an appointment or visit with a patient.

In some implementations, a GUI is configured to request and receive notes from a user, for example user observations, accomplishments, and/or setbacks related to pain, fatigue, mood, sleep, and/or overall wellbeing.

In some implementations, the application includes discussion board functionality, for example in private and public settings.

### Navigator Dashboard Graphical User Interfaces

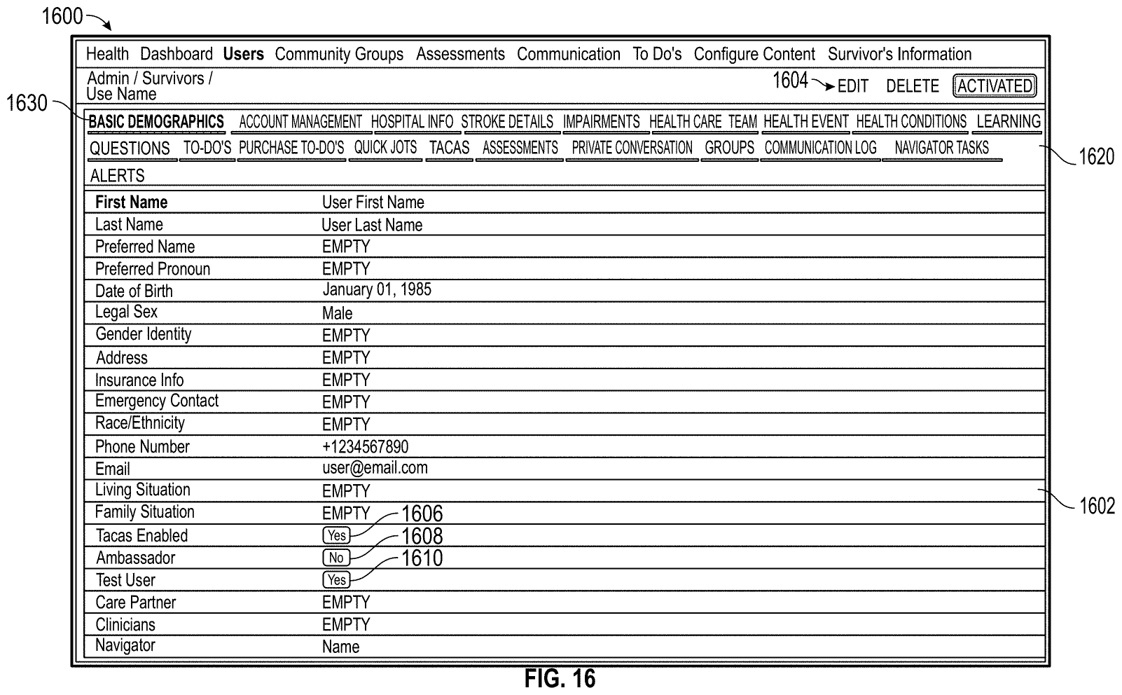

FIGS. 16-21 illustrate example navigator dashboard graphical user interfaces (GUI), according to some implementations of the present disclosure. In various implementations, aspects of the navigator dashboards may be rearranged from what is shown and described below, and/or particular aspects may or may not be included. As described herein, the navigator dashboard GUIs of FIGS. 16-21 can enable a navigator to interact with or plan the care of a patient and/or assist caregivers. The navigator can access the navigator dashboard configurations of FIGS. 16-21 on a navigator computing device. The navigator dashboard configurations of FIGS. 16-21 may share similar user interface elements and/or capabilities.

Often times, patients may be discharged from care with too much or too little information regarding their health event and the recovery process. In addition, caregivers may be assisting several patients at once in varying stages of recovery and/or have limited knowledge regarding the care of the patient. The navigator dashboard interfaces can provide a tailored experience for other users based on each of the patient's needs. By possessing the relevant information in one centralized location, navigators can search for relevant information quickly using the various tabs and ribbons of the system 170. Further, navigators can monitor a patient's progress, assign educational content and assessments, assist a patient with their questions, provide guidance, and much more.

In FIG. 16, an example implementation of a navigator dashboard GUI 1600 is depicted. A navigator can locate the GUI 1600 by selecting the “Basic Demographics” element 1630 found in the ribbon 1620. The GUI 1600 can include a demographics list area 1602 that can comprise patient specific parameters and fillable and/or selectable fields. The GUI 1600 can display all the relevant demographic information mentioned above in the preceding sections for a navigator to view when communicating with a user, for planning and/or assigning assessments or learning lessons, when creating to-do lists, when assisting users with appointments, and the like. The navigator can distribute and/or share the patient specific parameters with caregivers such as with the patient's doctors or at home care takers. Further, the demographic list area 1602 can be edited and/or updated in response to a user selection of an edit element 1604 by the navigator or another user with access to the GUI 1600. In some implementations, the demographic list area includes indication elements 1606, 1608, 1610 indicating whether the navigator selected certain parameters for the patient. Additionally or alternatively, the demographic information can be updated automatically by a system with new or replacement information as the patent specific parameters displayed in the GUI can be connected to a healthcare network of the client.

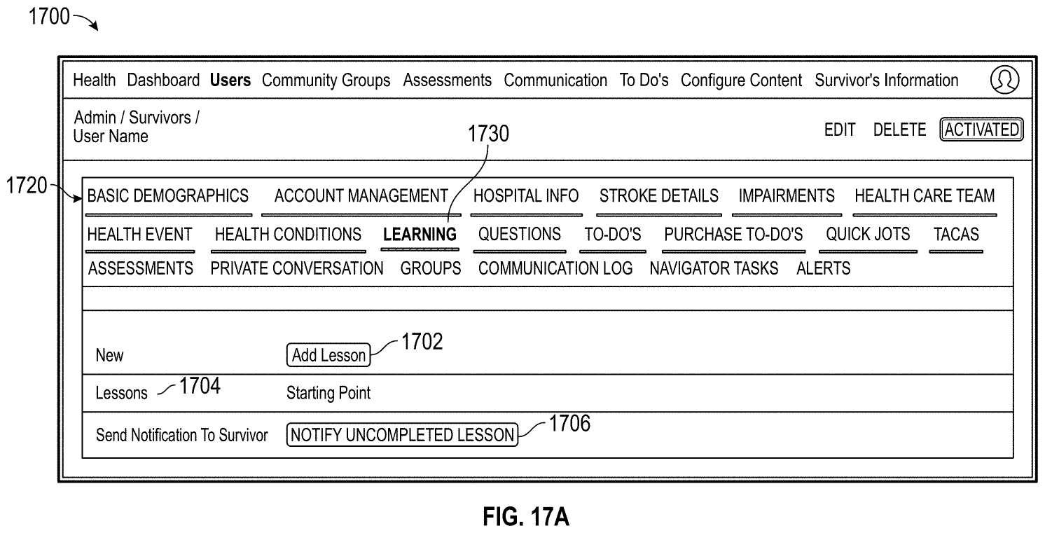

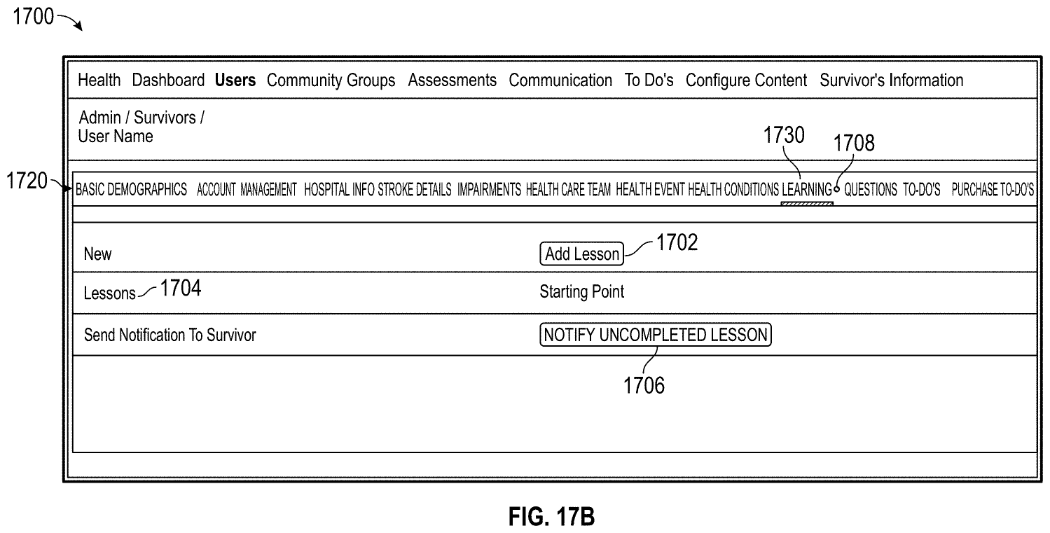

In FIG. 17A-17B, another example navigator dashboard GUI 1700 is depicted. A navigator can locate the GUI 1700 by selecting the “Learning” element 1730 found in the ribbon 1720. The GUI 1700 can allow a navigator to facilitate the patient's post-release education by assigning literary content, tasks, and the like. Using the GUI 1700, a navigator can assign a patient various learning lessons with the “Add Lesson” element 1702. In some implementations, the system and/or provides suggestions or assign various learning lessons at least based on partly on the specific information of the patient and/or the stage of recovery of the patient. In some implementations, the system and/or navigator provides learning content at least based on timing of the recovery process, time before and/or from discharge, and/or time of year. Each learning content can be identified by a reference number that can be used in tracking several different metrics for events before or after the user interacts with the learning content. Such metrics can include readership completion, compliance with the learning content, fall detection, indication of infection, time needed for completion, occurrence of events pre- and post-completion of the learning content, and the like. For example, by using metrics and identifying trends between potential health events and/or foreseeable experiences (e.g., developing a urinary tract infection of discharge from a care facility, falling, and going up and down stairs) and points in time (e.g., following discharge from a care facility, returning home, seasons), the system and/or navigator can assign learning content to the patient before the health event or experience occurs to assist in prevention or to inform. For example, an accelerometer in a user device can detect falls of the user and track such data. The data can then be used to develop and identify trends in the occurrence of such an event and assist in the assigning of articles related to falls. Further, the rate of falls can be measured following the assignment of fall articles which can be used to determine if user's rates are influenced after assigned such articles. In some implementations, the patient submits feedback to the system and/or navigator to improve optimization of the timing of release of content. In some implementations, the system collects data corresponding to patients completing learning content and generates trends between completion of learning content and health events. Such data can be used in determining trends between health events and compliance with assigned learning connect and whether compliance can improve or lessen the potential occurrence of certain health events.

Further, the navigator GUI 1700 can be configured to display various learning lessons and the corresponding title and tasks of the learning lessons in display area 1704. The learning lessons can be personally curated by a navigator towards a patient's specific health event or level of progress in the recovery stage. In some implementations, a patient and their caregiver are assigned similar learning lessons that appear differently to each user depending on the enrollee status of the user. In some implementations, the metrics related to the learning lessons are analyzed to determine trends in the recovery process and to assist in predicting events.

The navigator can also notify any of the corresponding users when the navigator assigns a new lesson, or to complete an uncompleted lesson, using the “Notify Uncompleted Lesson” element 1706. In some implementations, the system automatically submits notifications at least based on the date the learning lesson was assigned, recognition of a lack of activity, periodic reminders such as a daily or weekly notification, and the like. In some implementations, an algorithm assigns and populates new lessons at least partly based on the demographic data and medical history of the patient. In some implementations, the navigator views a completion or progress status of the learning lesson, the responses of the patient with respect to the learning lesson, and/or comments or notes from the patient related to the learning lessons.

FIG. 17B shows a notification alert 1708 after the completion of a learning lesson by a patient on their user computing device. The notification alert 1708 can be indicated by an icon in the dashboard ribbon 1720. For example, as shown in FIG. 17B, the alert may be a solid indicator of any shape or color adjacent to the “Learning” element 1730 in the dashboard ribbon 1720. In some implementations, the color of the notification alert 1708 indicates a level of priority such as a red notification indicating the highest priority alert and green notification indicating a lower priority. Further, once all learning lessons are completed, the “Notify Uncompleted Lesson” element 1706 may become locked such that the navigator and/or system is no longer able to send notifications to the patient.

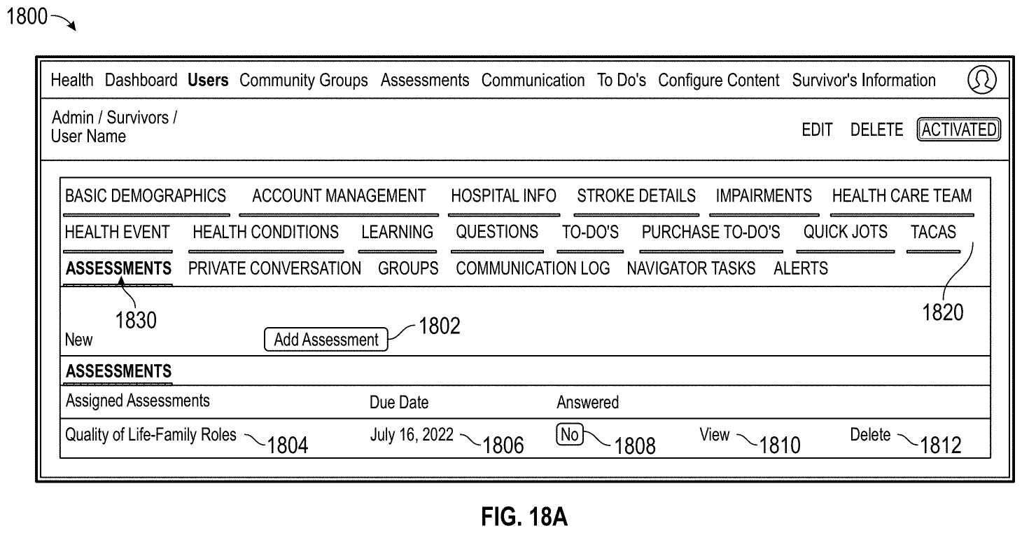

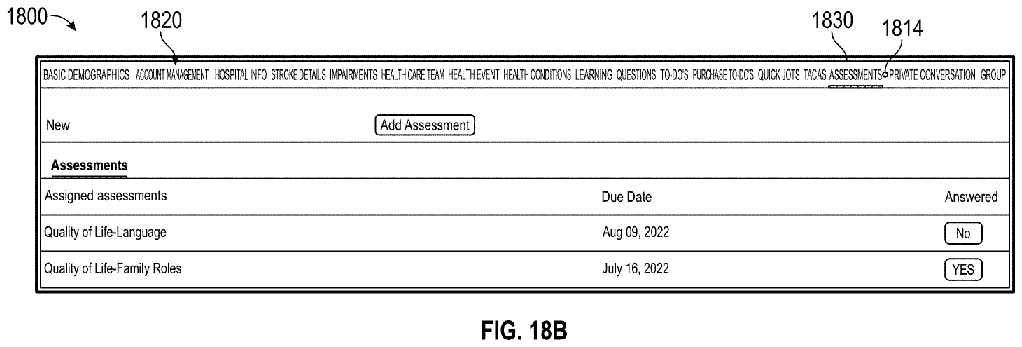

In FIG. 18A, another example implementation of a navigator dashboard GUI 1800 is depicted. A navigator can locate the GUI 1800 by selecting the “Assessments” element 1830 found in the ribbon 1820. In using navigator dashboard GUI 1800, a navigator can assign assessments for a user to complete using the “Add Assessment” element 1802. In some implementations, the navigator assigning an assessment pushes a notification to the user's device. The GUI 1800 can allow the navigator to gauge the progress of the patient based on the completion of the assigned assessments or the lack thereof. Further, GUI 1800 can allow the navigator to assign assessments based partly on the specific paraments of the patient and/or the stage of recovery of the patient. In some implementations, the system assigns new assessments based at least on the specific paraments of the patent and/or the stage of recovery of the patient. For example, a navigator and/or the system may assign a new assessment once the patient is discharged from a care facility in order to gauge the state of the patient. In other examples, the navigator and/or system may assign a daily, weekly, monthly, etc. assessment for monitoring purposes. The patient's responses can be used for assigning future assessments and monitoring the patient's progress.

Assessments can vary from a wide range of topics which can inform the navigator, for example, if the navigator, caregiver, or clinician should provide assistance, if a learning lesson should be assigned that covers topics related to an assessment, and/or if the navigator should further curate the types of assessments. In some implementations, the GUI 1800 is configured to is display an assigned assessment in a display area 1804 along with a due data 1806, a completion 1808 element indicating whether the patient answered or completed the assessment, a view element 1810 to view the assessment, and/or a delete 1812 element to delete the assigned assessment.

FIG. 18B shows a notification alert 1814 after the completion of an assessment by a patient on their user computing device. The notification alert 1814 can be indicated by an icon in the dashboard ribbon 1820. For example, as shown in FIG. 18B, the alert may be a solid indicator of any shape or color adjacent to the “Assessment” element 1830 in the dashboard ribbon 1820. In some implementations, the color of the notification alert 1814 indicates a level of priority such as a red notification indicating the highest priority alert and green notification indicating a lower priority.

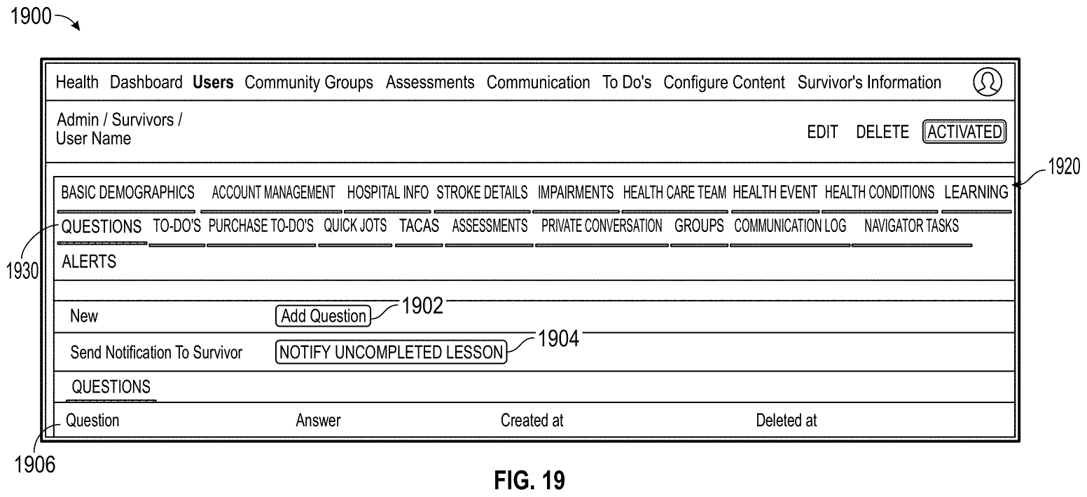

In FIG. 19, another example implementation of a navigator dashboard GUI 1900 is depicted. A navigator can locate the GUI 1900 by selecting the “Questions” element 1930 found in the ribbon 1920. In using navigator dashboard GUI 1900, navigator can suggest and add new questions for a user using the “Add Question” element 1902. The navigator can further send notifications and/or reminders to the user using the “Notify Uncompleted Questions to Ask” element 1904. The GUI 1900 can allow the navigator to submit topics for patients to ask during sessions with their caregivers such as nurses, doctors, therapists, and the like. In some implementations, the navigator dashboard GUI 1900 includes the display area 1906 which is configured to display questions selected by the patient and the corresponding answer provided by the patient, caregiver, and/or navigator. By viewing display area 1906, navigators can monitor a user's previously submitted or selected questions which can assist the navigator in suggesting new and/or alternative questions, responding to said questions, and/or providing or requesting assistance for the user (e.g., requesting medical assistance). In addition, the display area 1906 can assist the navigator in selecting assessments or learning lessons based at least in part on the patient's questions and answers.

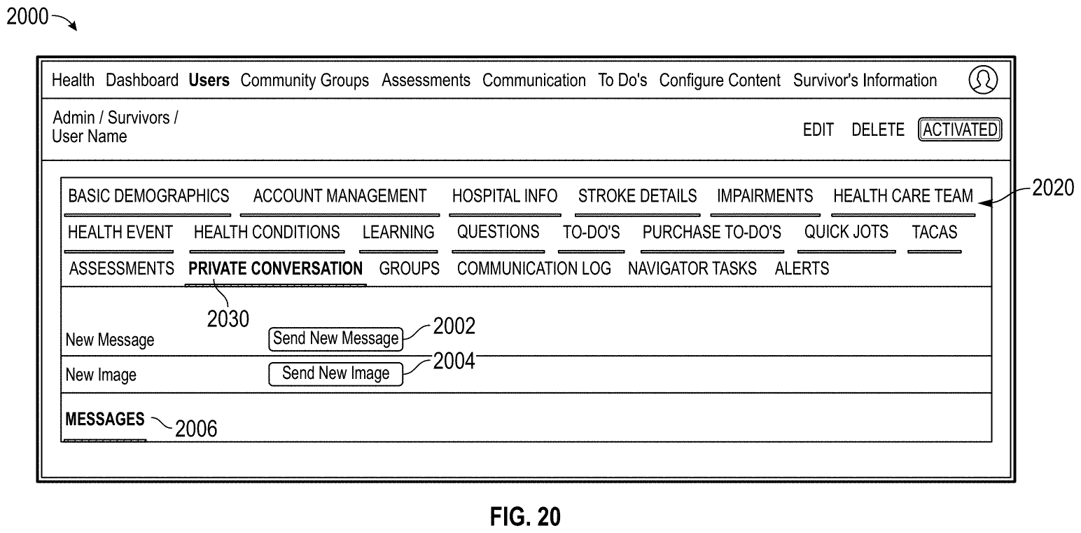

In FIG. 20, another example implementation of a navigator dashboard GUI 2000 is shown. A navigator can locate the GUI 2000 by selecting the “Private Conversation” element 2030 found in the ribbon 2020. GUI 2000 can facilitate communication between the navigator and the patient. The navigator dashboard GUI 2000 can allow a navigator to converse privately with a user, for example a patient, through private messages. Using the navigator dashboard GUI 2000, a navigator can send private messages by selecting the “Send New Message” element 2002. Further, the navigator can also send messages comprising of images by selecting the “Send New Image” element 2004. A display area 2006 can be configured to display the private messages between the navigator and the patient.

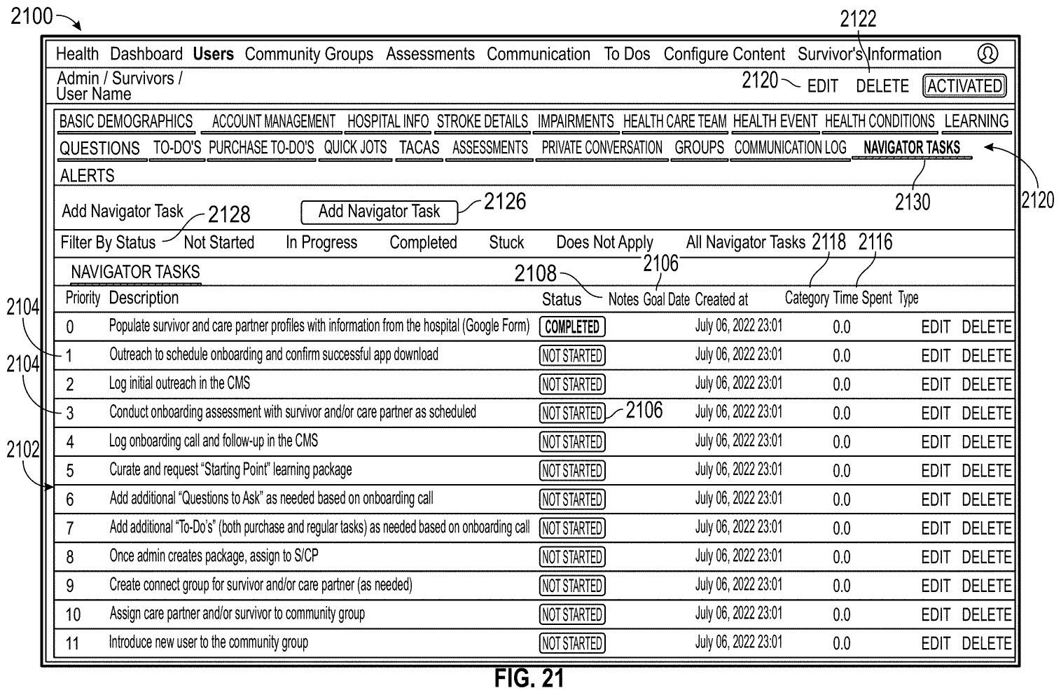

In FIG. 21, another implementation of a navigator dashboard GUI 2100 is depicted. GUI 2100 can assist managing the tasks and priorities of one or more patients assigned to a navigator. A navigator can locate the GUI 2100 can by selecting the “Navigator Tasks” element 2130 found in the ribbon 2120. The GUI 2100 can include a list 2102 of tasks for a navigator to complete which can include tasks designed to assist a user and/or administrative tasks. Further, the list 2102 can include several fields such as a priority indicator 2104 which indicates the level of priority assigned to a navigator task, a status element 2106 indicating the progress of any one of the navigator tasks, a notes display 2108, a goal date display 2110, a navigator task creation display 2112, a category display 2114, a time spent indicator 2116, and/or a type of navigator task indicator 2118. Further, a navigator can edit the GUI 2100 with edit element 2122 and delete assigned tasks with delete element 2124. In some implementations, the tasks in list 2012 is configured to be dragged and dropped to edit prioritize list 2102. The GUI 2100 can assist the navigator with maintaining the patient's curated program and ensure that the patient receives the necessary care. For example, the navigator can add tasks to the list 2102 by selecting the “Add Navigator Task” element 2126 and adding the desired task. Additionally or alternatively, the GUI 2100 can provide the navigator with filter elements 2128 to enable the navigator to filter the tasks contained in the list 2102.

### Mobile Application Graphic User Interface

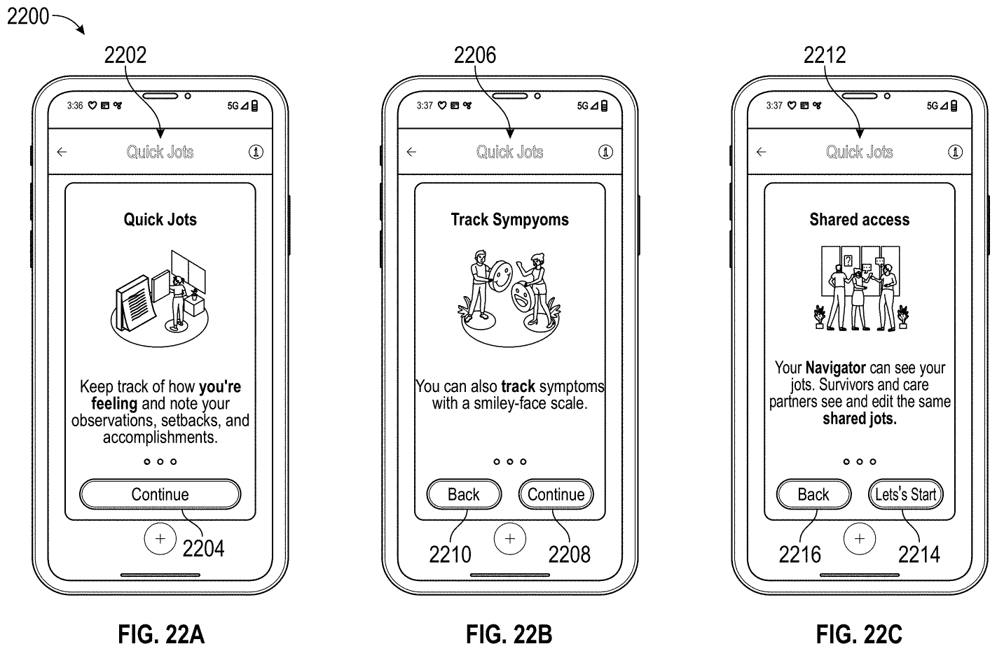

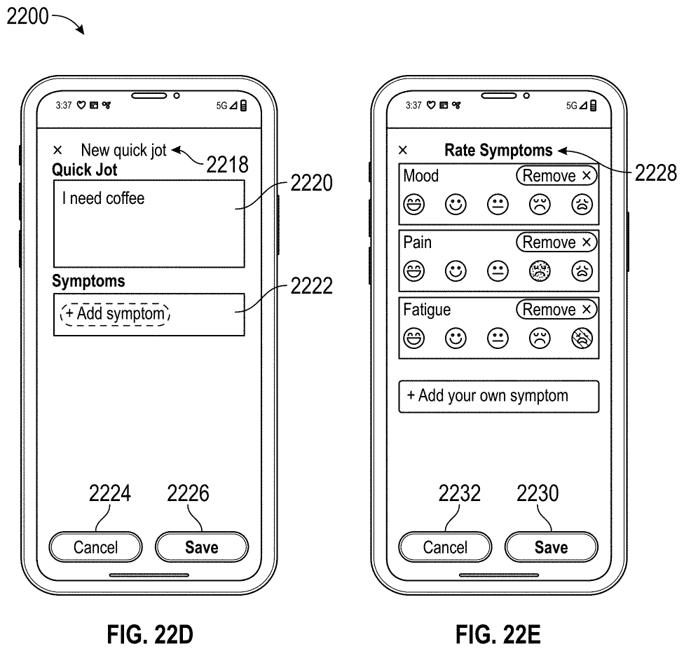

FIGS. 22A-30 illustrate example mobile application graphical user interfaces (GUI), according to some implementations of the present disclosure. In various implementations, aspects of the mobile application may be rearranged from what is shown and described below, and/or particular aspects may or may not be included. As described herein, the mobile application GUIs of FIGS. 22A-30 can enable a user to interact with a post-health event care program. The user can access the mobile application graphical configurations of FIGS. 22A-30 on a user device. The mobile application configurations of FIGS. 22A-30 may share similar user interface elements and/or capabilities.

Through the use of a mobile application, users can participate in a post-health event care program. The mobile application can provide an assortment of aids such as learning lessons, assessments, facilitation, communication, requesting assistance, and many more. Once the user is approved to participate in the program, the user can download the mobile application to their user device for accessing.

FIG. 22A-22E shows five views of a mobile application note taking process. For example, FIG. 22A illustrates an example mobile application GUI 2200 showing a prompt screen 2202 for a user to record their emotional and physical state within the mobile application. The mobile application can request the user to input, for example, their own observations, setbacks, accomplishments, and the like by selecting the “Continue” button 2204. FIG. 22B shows a screen display 2206 configured to display a message to the user to track symptoms using methods such as a graduated scale or the like. The user may proceed by selecting the “Continue” button 2208 or return to the previous page by selecting the “Back” button 2210. FIG. 22C illustrates an example display of an information screen 2212 directed towards the user that shared information can be viewed by a navigator. Additionally or alternatively, users can see and edit the shared notes in the application. Before recording, the user can have an option to proceed by selecting the “Continue” button 2214 or returning to the previous page by selecting the “Back” button 2216. FIG. 22D illustrates an example “Quick Jot” screen 2218 for a user to record notes in blank text field 2220. A user can record notes by typing into the text field 2220, recording an audio message, and/or downloading a picture, image, and/or video file, Additionally or alternatively, a user can select the “Add Symptoms” button control 2222 to populate one or more symptoms within the “Quick Jot” screen 2218. The user can complete the screen by selecting a “Cancel” button 2224 to discard changes or a “Save” button 2226 to save user's input. Lastly, FIG. 22E illustrates an example Rate Symptoms screen 2228 for a user to record the characteristics of the experienced symptoms, such as, mood, pain, fatigue, and the like comprising a graduated scale or similar method. The graduated scale can be shown using numerals, images, color changes, etc. Often after experiencing a stroke, patients may not be able to process scales in manner that can be communicated effectively due to a health event. In such situations, patients can instead associate certain images and/or colors with their thoughts and/or feelings. Additionally or alternatively, the patient may need to configure a scale display for accessibility. For example, instead of using numerals or text, the user can update the graduated scale to contain images and/or colors (e.g., using a series of emoticons depicting facial expressions or a series of icons changing colors). The images may be from a pre-selected bank of images, or the user can upload images. In some implementations, the user uploads one or more personalized images and/or icons to create the graduated scale. The graduated scale or similar method may include icons that indicate levels of intensity the user is experiencing. In some implementations, the user can add new symptoms as the user experiences such symptoms. The new symptoms can appear above, between, and/or below the previously displayed symptoms. The newly added symptoms can contain a new graduated scale configured to be interacted with the user. The user can then save the symptoms rating by selecting a “Save” button 2230 or can exit screen 2228 by selecting a “Cancel” button 2232.

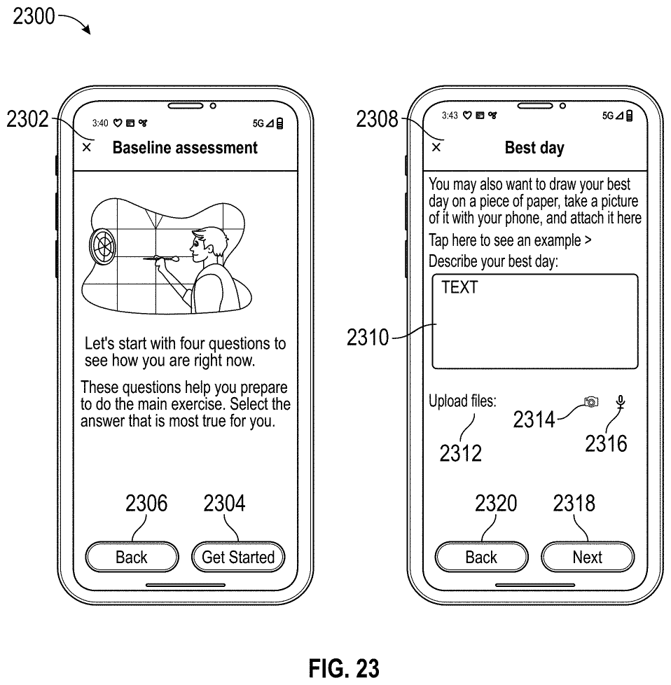

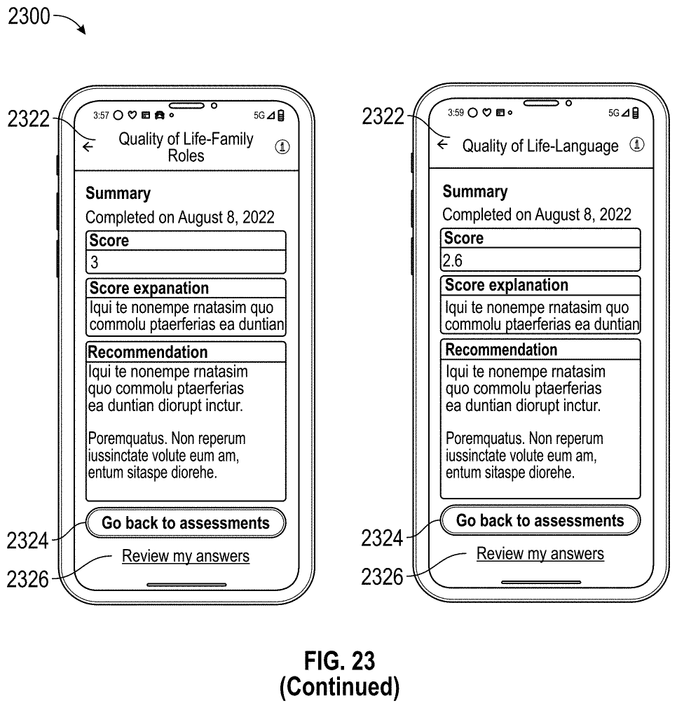

FIG. 23 illustrates four views of an example mobile application GUI 2300 showing an assessment process. A navigator can assign assessments for a user to complete on their user computing device. For example, FIG. 23 shows an implementation of an “Assessment Main” screen 2302 that could be assigned to a user. An assessment view can be configured to display relevant descriptions associated with the assigned assessment. Such descriptions can include information regarding the assessment including the number of questions or purpose of the task. From the screen 2302, the user can progress to the assessment by selecting a “Get Started” button 2304 or return to a previous screen by selecting a “Back” button 2306. The “Assessment” screen 2308 can illustrate an example of an assessment questions. Using screen 2308, a user can respond to the presented by typing a response into a text field 2310, upload the response file (e.g., picture, video, sound, text file, etc.) using the “Upload files:” button 2312, take a picture or video by selecting a “Camera” button 2314, and/or record a voice response by selecting a “Microphone” button 2316. Once the user completes a response, the user can proceed to the next screen by selected the “Next” button 2318 or return to the previous screen by selecting the “Back” button 2320. “Summary” screens 2322 can illustrate a result from completing the “Assessment” screen 2308. The “Summary” screens 2322 can provide feedback and insight into the user's responses. Addition or alternatively, the “Summary” screens can provide feedback such as a score, an explanation coupled to the score, recommendations at least based on the score, and the like. In some implementations, navigators assign learning lessors or further assessments based partially on the score received in the assessment. Further, the navigator can adjust the patient's curriculum or recovery process based on the received score. The user can continue back to the “Assessment Main” screen 2302 by selecting the “Go back to assessments” button 2324. Further, the user can review their answers by selecting the “Review my answer” button 2326.

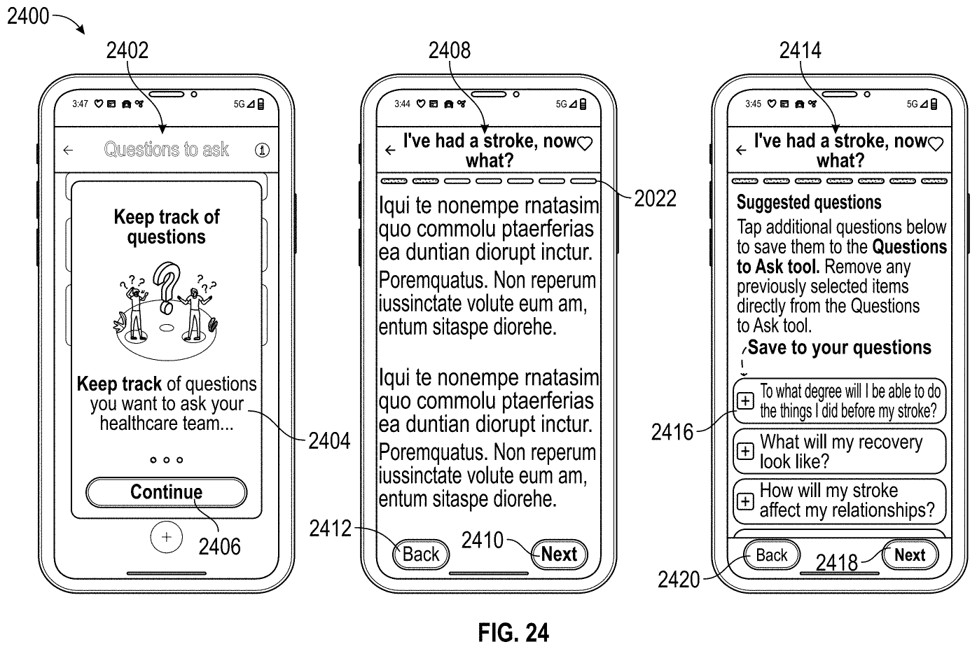

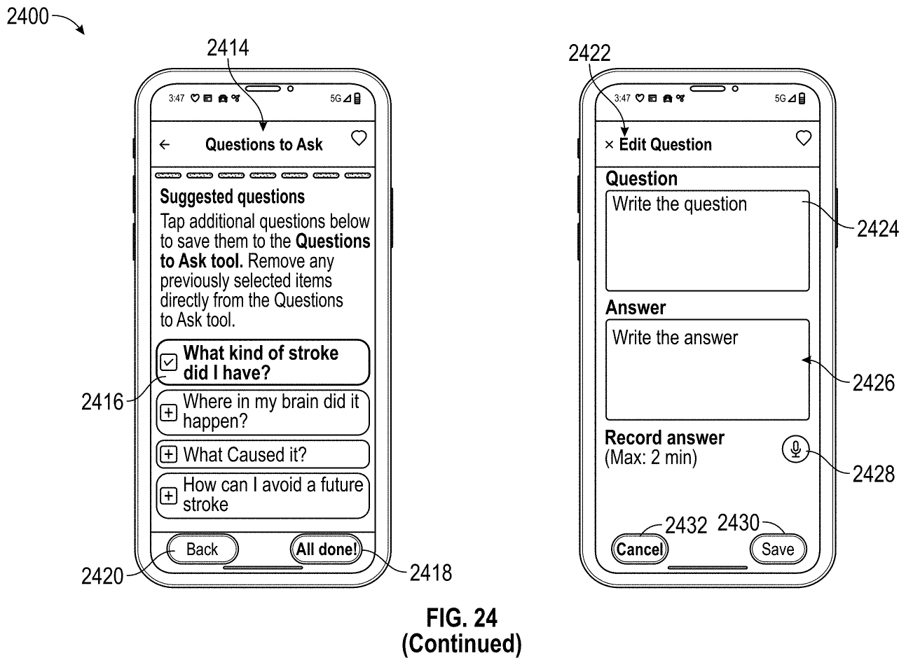

FIG. 24 illustrate views of an example mobile application GUI 2400 showing a question process that can allows a user to keep track of various question. A user, such as a patient, caregiver, and/or navigator, can save, record, and/or submit questions to the mobile application for storage. A user can be reminded and/or prompted to keep track of various questions by a reminder screen 2402. Example reminder screen 2402 can follow learning lessons, assessments, to-do's, and/or any content in the mobile application. The first screen can be configured to display a message 2404 suggesting the user keep track of questions to ask their healthcare team. To continue to the next scree, the user can select a “Continue” button 2406. The user can then proceed to an education screen 2408 which can include general information regarding strokes. The education screen 2408 can provide general information and assist the user in preparing questions. The user can then select the “Next” button 2410 to proceed to the next screen or return to the previous screen by selecting the “Back” button 2412. An additional “Suggested questions” screen 2414 can be configured to display a list 2416 of suggested questions for the user to ask. The user can tap one or more of the questions from list 2416 shown in screen 2414 to save the selected question. Once the user has completed selecting or not selecting questions from list 2416, the user can proceed to the next screen by selecting an “All done!” button 2418 or “Back” button 2420 to return to a previous screen. Additionally or alternatively, the question process can include an “Edit Question” screen 2422 which can include an editable text field 2424 such that the user can record personalized questions to ask. Further, screen 2422 can include an “Answer” text field 2426 to record responses to question saved in the field 2424. The user can also record an audio answer by selecting the “Microphone” button 2428. In some implementations, the answer to the question is transcribed on the user device. Once the user completes the question process, the user can close the process by selecting a “Save” button 2430 to save responses or a “Cancel” button 2432 to return to a previous screen. Further, saved answers can be accessed by the navigator.

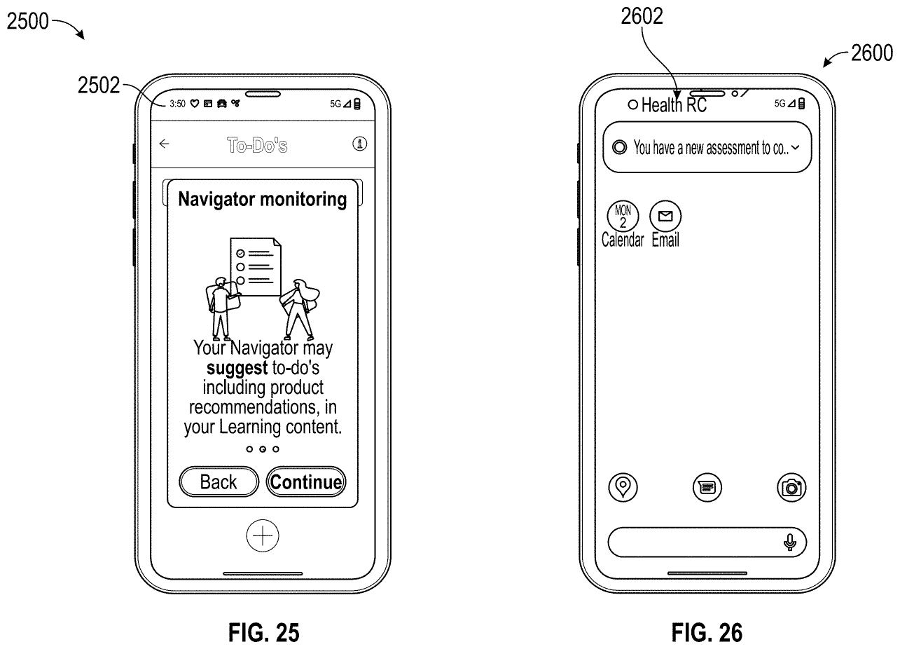

FIG. 25 illustrates different views of an example mobile application GUI 2500 showing a navigator monitoring message. For example, “Navigator monitoring” screen 2502 can notify a user that a navigator may suggest “to-do's” which can include product recommendation. These items can be saved in the user's learning content for the user to consider. Further, the navigator can create to-do lists associated with other elements of the mobile application or that can include tasks outside of the application.

FIG. 26 illustrates a view of an example mobile application GUI 2600 showing a notification on a user device 120. A notification 2602 can appear on any operating system as an alert such as a text, pop-up alert, instant message, and the like. A user can select the notification or a link contained in the notification to open the mobile application. In some implementations, selecting the notification takes the user directly to the page within the mobile application that triggered the notification.

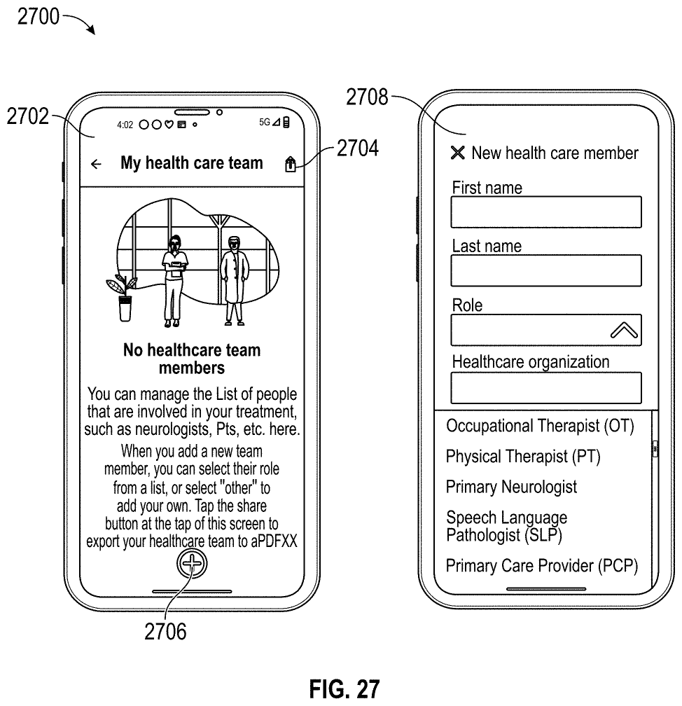

FIG. 27 illustrates two views of an example mobile application GUI 2700 showing a mobile application display of a user's healthcare team members. For example, a “My health care team” home screen 2702 can be configured to display general information related to the healthcare team members. Further, the user can select a “Share” button 2704 to export the healthcare team member. The user can add new health care members by selecting the plus button 2706. The user can input new healthcare members using a “New health care member” screen 2708. For instance, the user can input a first, name, last name, role, healthcare organization, and the like. In some implementations, new healthcare team information is automatically retrieved from a remote computing device.

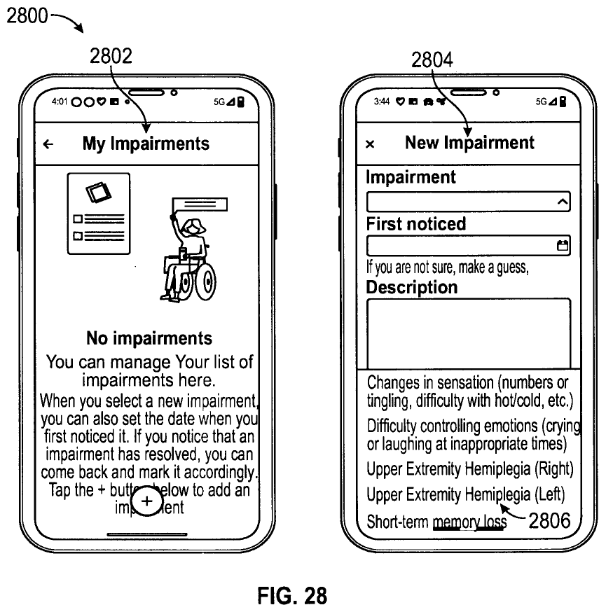

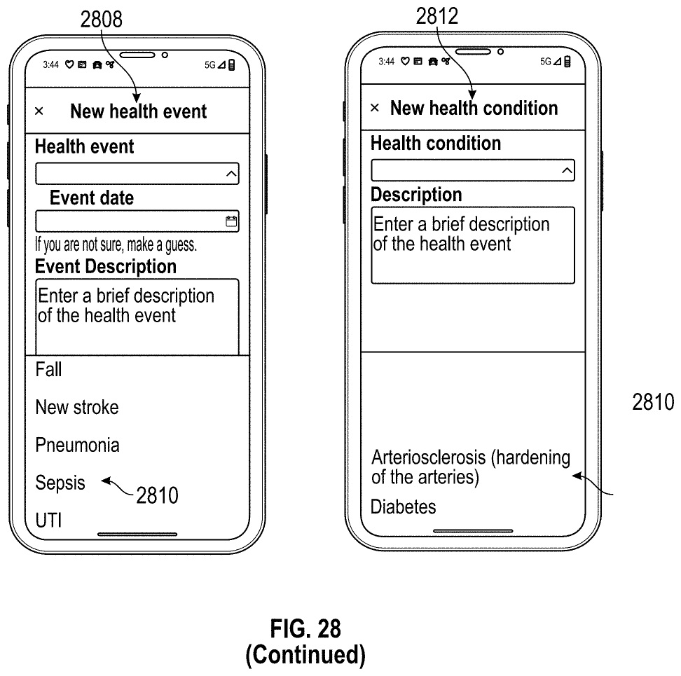

FIG. 28 illustrates four views of an example mobile application GUI 2800 showing a mobile application impairment process. An impairment home screen 2802 can be configured to display general information and directions to a user for managing their list of impairments. The user can record impairments using the new impairments screen 2804. When recording a new impairment, the user can record the impairment, when the user first noticed the new impairment or a guess of when the impairment first began, a description of the impairment, and the like. A list of impairments 2806 can be included in the impairment name field such that a user can select a predefined impairment from the drop-down menu. In some implementations, the user records new health events using a new health event screen 2808. For example, the user can record a name, event date or guess of an event date, an event description, and the like. Additionally or alternatively, a list of health events 2810 can be included such that the user can select a preselected health event. The user can further record new health conditions using the new health condition screen 2812. The user can record a name of the health condition, a description of the health condition, and the like. Further, the user can select a predefined health conditions from a list of health conditions 2814.

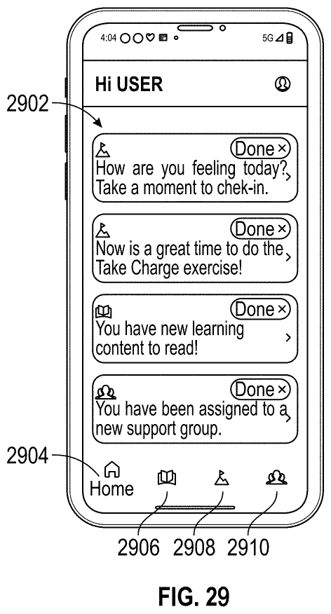

FIG. 29 illustrates an example of a mobile application home screen. From the home screen 2902, a user can open the mobile application to the home screen 2902 where the user can view various elements including tasks, learning content, assessments, general information, messages from a navigator, and the like. The user can also select one of several elements such as a home button 2904, learning content button 2906, a “Accomplish” button 2908, a “Connect” button 2910, and the like. In some implementations, the home screen elements, such as the elements mentioned above, appear automatically based on user interactions with the system. For example, a message element can appear after a navigator submitted the message to the user. In some implementations, the user can mark the element as “done” to dismiss the elements from the home screen.

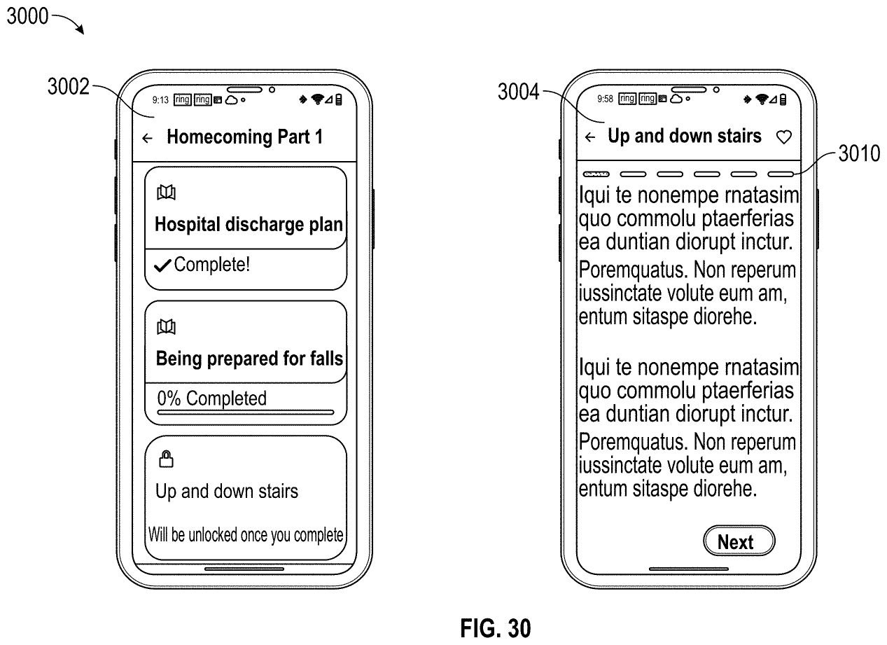

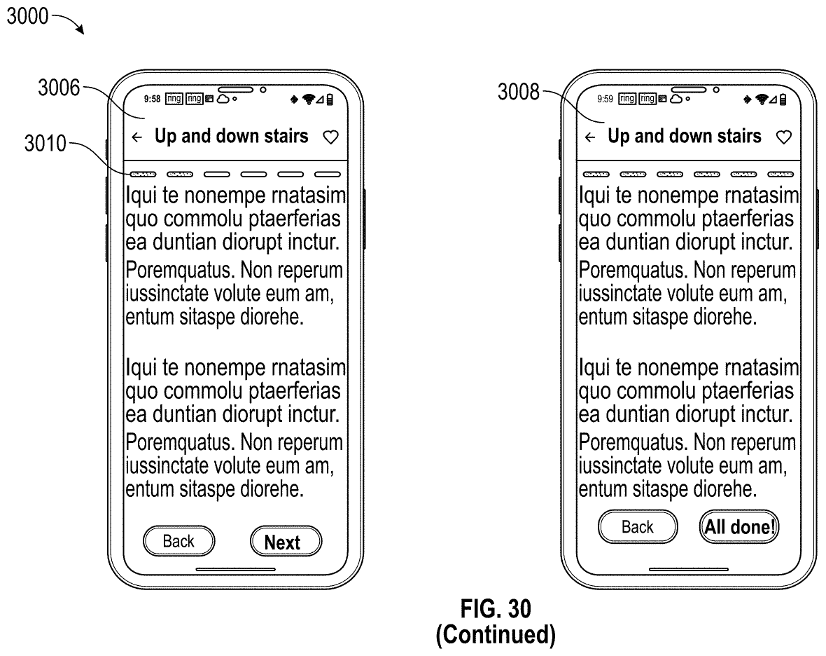

FIG. 30 illustrates four views of a mobile application GUI 3000 displaying examples of timing of content delivery. In some implementations, content is selected and/or displayed in the GUI to the user based on time appropriateness. For example, time appropriateness may be based on at least an amount of elapsed time from the health event; a projected health trajectory based on a severity of the health event; a prescribed treatment or therapy; a history of the patient; and/or symptoms and/or disabilities experienced by the patient. For example, screen 3002 can display relevant information and a learning lesson for a patient that is associated with a point in time in the recovery process (e.g., the user receiving content manually and/or automatically after discharging from a care facility). In some implementations, the screen 3002 is automatically updated based on assigned content. In some implementations, the timing of content delivery depends on the completion of modules or tasks. For example, the user may receive content at least based on actions in the mobile application such as identifying fatigue as an impairment and/or as a response to an assessment. A learning content may have an indication informing the user that the learning content is locked until the user performs a task and/or the navigator unlocks the learning module. The system may also unlock the disabled learning module automatically following the completion of an interaction by the user. The user may then receive manually and/or automatically a learning lesson in response to the interaction. A system can also automatically schedule the timing of content delivery, or a navigator can schedule the timing of content delivery at least based on the navigator's observations of the patient or stage in the recovery process. For example, screens 3004, 3006, and 3008 can display relevant learning lessons associated with a stage in the recovery process. In some implementations, the learning content is related to experiences or potential health events the patient may potentially encounter. In some implementations, a notification is sent to the to the patient user device to inform the patient of assigned learning contents. In some implementations, GUI 3000 includes page indicators 3010 configured to indicate the screen and/or page the user is viewing. The page indicator can be contrasted against other page indicators 3010 on GUI 3000 to show which screen and/or page is selected. In some implementations, the distinct pages can be based on at least the user's impairment.

### Methods for Learning Content Review Process

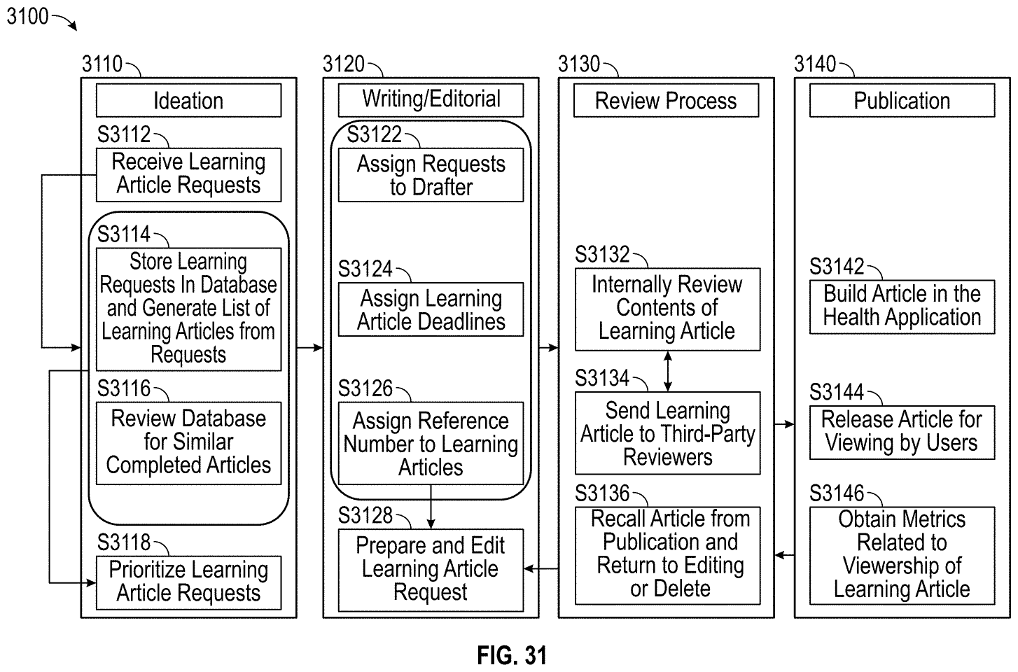

FIG. 31 shows a flow diagram corresponding to example content creation and distribution system 3100 for learning content management such as a learning content review process. System 3100 can be implemented by the systems mentioned herein. Further, system 3100 can be an all-encompassing process containing all the tools and resources for the learning content management process. For example, the system 3100 can replace, for example, several programs requiring different file types, needing to download and/or upload files, separate processes not in communication with one another with a more streamlined system. For example, system 3100 can improve content management by including the different stages mentioned herein in one location such that editors, reviewers, publishers, and the like can transition between the different stages without exiting system or needing to change to programs. Additionally or alternatively, system 3100 can allow for drafting, editing, reviewing, and publishing all from one system without necessitating the downloading of local copies corresponding to the learning content. For illustrative purposes, the system 3100 will be described as being implemented by components of computing environment 100 of FIG. 1. The system 3100 depicts an example overview of reviewing learning content. In general, the system 3100 may include various stages, for example an ideation stage 3110; a writing/editorial stage 3120; a content review stage 3130; and a publication stage 3140. The system 3000 can be used to create learning articles, route said articles for approval, assign and track the learning content via document number, assign the learning content to patients, track the progress of the learning articles, and edit and recall the learning articles for updating.

Any process descriptions, elements, or blocks in the flow diagrams described herein and/or depicted in the attached figures should be understood as potentially representing modules, segments, or portions of code which include one or more executable instructions for implementing specific logical functions or steps in the process. Alternate implementations are included within the scope of the implementations described herein in which elements or functions may be deleted, executed out of order from that shown or discussed, including substantially concurrently or in reverse order, depending on the functionality involved, as would be understood by those of ordinary skill in the art.

Turning now to ideation stage 3110, which includes one or more of: receiving learning article requests at block 53112; storing learning articles in database and generating a list of learning articles at block 53114; review database for similar completed articles at block 53116; and/or prioritizing learning article requests 53118. Anyone of the type of users can submit learning article requests to the content management system for consideration. Following the receipts of one or more requests, the learning content management system can prioritize the requests at block 53118 at least based on the requestee, a keyword search, and/or population served. For example, users such as medical professionals can request learning articles to provide patients with information in response to commonly asked questions. While any user can submit a learning article request, requests from medical professions may be prioritized above certain others. Additionally or alternatively, the click rate, reading count, key word searching of learning articles can prompt the generation of similar articles to address similar issues to further aid in the transition following a health evening. One or more similar requests may be made over time showing a need for learning article topic. Trends in requests can assist the system in determining user needs.

In some implementations, as shown at block 53122 of stage 3120, the one or more requests from ideation stage 3110 are assigned for drafting. For example, block 53122 can include assigning a learning articled to written. In some implementations, articled can be assigned based on availability of the writers. In other implementations, the articles can be assigned based on the professional expertise of the writer and/or the writer's educational background. Along with assigning a learning article request to a writer, a deadline to produce the learning articled can be assigned at block 53124.

At block 53126, learning articles assigned for drafting can be assigned a reference number. In some implementations, the reference number can be used to track the progress of the learning article, to track the learning article for editing, and/or to recall the learning the article. Once the learning article request is assigned a drafter, deadline, and/or reference number, the drafter can begin preparing and editing the learning article as shown in block S3028. For example, a drafter can begin preparing a new learning articled at least based on an unmet need and/or a request for a new topic or category. In other implementations, a drafter can, for example, edit articles to update them based on new learnings or information. In cases of updating articled, drafters can use the reference number to retrieve previously prepared articles for revisions.

In some implementations, as shown at stage 3130, the learning article can be reviewed internally as in block 53132 or externally by third-party reviewers in block 53034. For example, as shown in block 53132, an internal reviewer may review the article to determine whether the learning articled is responsive to the learning articled request, that the learning article is properly formatted, and/or whether the learning article satisfies accessibility standards. During the internal review, the learning article can be assigned to content reviewers who specialize in certain topics. Learning articles can be edited in response to the internal review by the same learning article drafter in stage 3120 or a new drafter.

At block 53134, the learning articles can be distributed to third-party reviewers. Third-party review can include, but are not limited to, caregivers, stroke survivors, medical professionals, and the like. Third-party reviewers can determine the accessibility of the learning articles before publication. Further, if the learning articled is medically related, medical professionals can provide a quality review.

At block 53134, the learning articles recalled based at least partially on the assigned reference number. The learning articled can be recalled from publication to be updated, deleted, hidden, and/or the like. In some implementations, recalling articles deletes a local copy stored on a user device.

Following the review process, the learning article is published at stage 3140. The learning article can be built in the health application mentioned herein at block 53042. Feedback gleaned from stage 3130 can be considered in building the article in the health application. The learning article can be stored on a remote computing server or downloaded and stored on the user device. Once the build is completed, the learning article can be released to users at block 53144. Over time, metrics related to the viewership can be collected and processed at block 3146. The metrics can be used to improve learning articles request and provide feedback for further edits and recalls.

### User Post-Health Event Progression

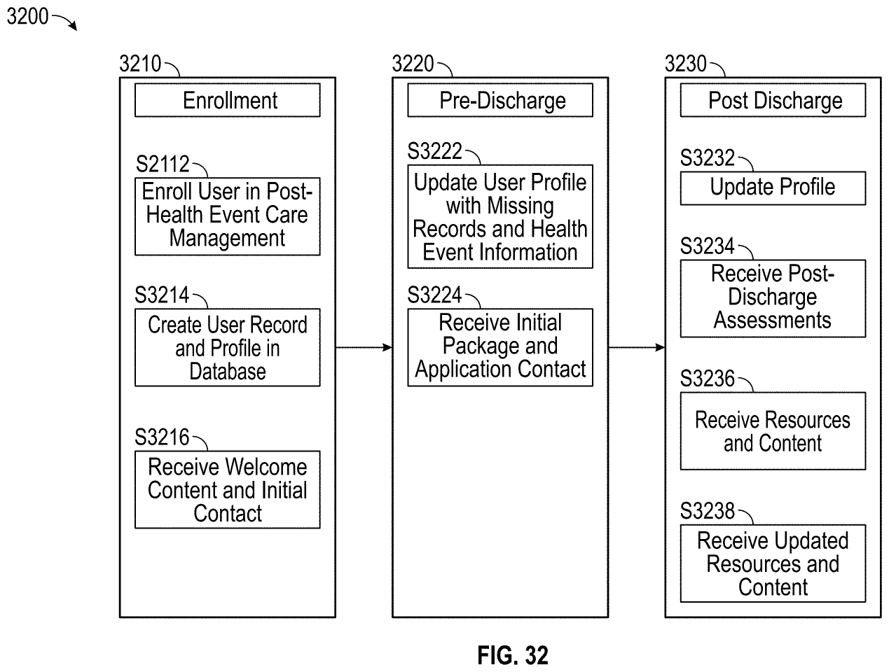

FIG. 32 shows a flow diagram corresponding to an example user post-health event progression 3200 which can illustrate the progress of a user following a post-health event. Progression 3200 depicts an example overview of the stages a user can experience overtime. In general, the progression 3200 can include: an enrollment stage 3210, a pre-discharge stage 3220, and post-discharge stage 3230. The progression 3200 can be used to track a user's recovery process following pre- and post-discharge from a care facility.

Turning to enrollment stage 3210, which can include: enrolling user in a post-health event care management program at block 53212; creating user record and profile in a database at block 53214; and/or a user receiving welcome content and an initial contact with the post-health even care management program at block 53216. The enrollment process at block 53212 can include a user being selected to participate in a post-health event program. A user can begin enrolment between admission to a care facility following a health event and prior to discharge. In some implementations, enrollment may not follow a health event and instead begin following a recommendation from a caregiver. Enrollment at block 53214 can include creating a user profile and updating the profile with the relevant medical records. A user's information can be uploaded into a database using the hospital medical records and self-reported information. Following the creation of a user profile, the user can receive introductory information and an initial contact at block 53216. The user can download an application associated with the post-health event care management program and begin reviewing any welcome content available.

Turning to the pre-discharge stage 3220, which in some instances may overlap with the enrollment stage 3210, any missing records and additional health event information can be further identified and collected to update the user profile at block 53222. A user can then receive an initial curated package and application content at block 53224 that can be at least partly based on the initial records. In some implementations, a post-care health program automatically selects the initial content from the medical records collected in blocks 53212 and 53222. Before the user is discharged from the care facility, the user can begin reviewing assigned learning content.

In some implementation, as shown at the post-discharge stage 3230, the user can report profile updates at block 53232. For example, the user may submit impairments, health events, additional records, and/or living situation. Following discharge, the use can also begin receiving post-discharge assessments at block 3234. Such assessments can be directed towards the physical and emotional state of the user. In some implementations, the assessments can be based on the severity of the health event and/or the level of impairment a patient is experiencing. A patient can receive assessments and learning content directed towards their situation or more general content. At block 53236, as the user progresses through the recovery process, the user can receive resources and learning content at least based on the progression and recovery rate of the user. For instance, a navigator may assign content that reflects improvement or setback to further curate the patient's experience. In some implementations, the results from the assigned content and learning experience is reported back to the care facility for determine courses of action. In the post-discharge stage 3230, the patient can continuously receive content from the navigator at least based on the recovery process. Content assigned to a user discharged from a care facility, such as a hospital, can differ from the content a user further along the recovery process could be assigned. As the user progresses through the recovery process, the user's state can change which influences the recovery program. For example, as the user completes learning lessons, the navigator and/or system may assign content that corresponds to the state of recovery such as more complicated exercises or returning to work. Additionally or alternatively, the GUI of a user computing device can further update to reflect the state of the user during the recovery process following discharge from a care facility. For example, the GUI may update to increase user interaction with the user device or alter the display of the content on the user device.

### Terminology

The foregoing is a summary, and thus, necessarily limited in detail. The above-mentioned aspects, as well as other aspects, features, and advantages of the present technology will now be described in connection with various implementations. The inclusion of the following implementations is not intended to limit the disclosure to these implementations, but rather to enable any person skilled in the art to make and use the contemplated invention(s). Other implementations may be utilized, and modifications may be made without departing from the spirit or scope of the subject matter presented herein. Aspects of the disclosure, as described and illustrated herein, can be arranged, combined, modified, and designed in a variety of different formulations, all of which are explicitly contemplated and form part of this disclosure.

In general, as used herein, a “user” may include, but not be limited to, a patient, a caregiver, a care partner, a healthcare provider, a navigator (e.g., user that helps or enables system setup), a friend, a relative, a family member, a nurse, a support group member, a therapist, a service provider, etc.

In general, as used herein, a “health event” may include a stroke, a traumatic brain injury, a multiple sclerosis relapse, episode, or diagnosis; a Parkinson's Disease diagnosis, a Diabetes diagnosis; a hypo or hyperinsulinemia episode; a Fibromyalgia diagnosis; a Cervical spondylosis episode or diagnosis; a Guillain-Barre syndrome diagnosis or episode; a Lambert-Eaton myasthenic syndrome diagnosis or episode; a Myasthenia gravis episode or diagnosis; an amyotrophic lateral sclerosis diagnosis; a spinal cord injury; or any other event, condition, or disease that causes neurological changes, disruptions, or loss of function or disruption or muscle weakness, spasticity, or loss of function.

In general, as used herein, “symptoms” of stroke onset or “disabilities” as a result of a stroke event may include, but not be limited to: blurred vision; speech impediments; slurring speech; involuntary eye or other body part movement; memory changes; balance changes; hemiplegia; gait changes; motor activity changes; muscle stiffness; muscle spasticity; behavioral changes (e.g., anxiety, anger, irritability, lack of concentration, lack of comprehension, etc.); emotional changes (e.g., depression); shoulder pain; shoulder subluxation (i.e., partial shoulder joint dislocation); altered smell, taste, hearing, and/or vision; drooping of eyelid (i.e., ptosis); weakness of ocular muscles; decreased reflexes; decreased sensation and muscle weakness of the face; nystagmus; altered breathing rate; altered heart rate; weakness in tongue; weakness in sternocleidomastoid muscle; aphasia; dysarthria; apraxia; visual field defects; hemineglect; disorganized thinking; confusion; hypersexual gestures; lack of insight of his/her disability; vertigo; disequilibrium; lack of consciousness; headache; vomiting; etc.

As used herein, user input received into the system may be either static or dynamic. For example, a demographic of a user may be static (e.g., requiring infrequent updating), while a biometric of a user may be dynamic (e.g., requiring frequent updating).

Dynamic user input, either collected directly by the software, a device communicatively coupled to the software, or a user of the software, includes but is not limited to: living situation, financial situation, clinical data (e.g., vitals, lab test results, scans, assessments, health history, etc.), recent assessments (e.g., neurological, motor skills, cognitive, etc.), identity, goals (e.g., be able to drive again, regain a motor skill, etc.), disabilities related or unrelated to the stroke (e.g., aphasia, dysphagia, fatigue, incontinence, etc.), life events, life changes, etc.

The systems and methods of the preferred embodiment and variations thereof can be embodied and/or implemented at least in part as a machine configured to receive a computer-readable medium storing computer-readable instructions. The instructions are preferably executed by computer-executable components preferably integrated with the system and one or more portions of the hardware processor on the user device, wearable, and/or computing device. The computer-readable medium can be stored on any suitable computer-readable media such as RAMs, ROMs, flash memory, EEPROMs, optical devices (e.g., CD or DVD), hard drives, floppy drives, or any suitable device. The computer-executable component is preferably a general or application-specific hardware processor, but any suitable dedicated hardware or hardware/firmware combination can Additionally or alternatively execute the instructions.

As used in the description and claims, the singular form “a”, “an” and “the” include both singular and plural references unless the context clearly dictates otherwise. For example, the term “input” may include, and is contemplated to include, a plurality of inputs. At times, the claims and disclosure may include terms such as “a plurality,” “one or more,” or “at least one;” however, the absence of such terms is not intended to mean, and should not be interpreted to mean, that a plurality is not conceived.

The term “about” or “approximately,” when used before a numerical designation or range (e.g., to define a length or pressure), indicates approximations which may vary by (+) or (−) 5%, 1% or 0.1%. All numerical ranges provided herein are inclusive of the stated start and end numbers. The term “substantially” indicates mostly (i.e., greater than 50%) or essentially all of a device, substance, or composition.

As used herein, the term “comprising” or “comprises” is intended to mean that the devices, systems, and methods include the recited elements, and may additionally include any other elements. “Consisting essentially of” shall mean that the devices, systems, and methods include the recited elements and exclude other elements of essential significance to the combination for the stated purpose. Thus, a system or method consisting essentially of the elements as defined herein would not exclude other materials, features, or steps that do not materially affect the basic and novel characteristic(s) of the claimed disclosure. “Consisting of” shall mean that the devices, systems, and methods include the recited elements and exclude anything more than a trivial or inconsequential element or step. Implementations defined by each of these transitional terms are within the scope of this disclosure.

The examples and illustrations included herein show, by way of illustration and not of limitation, specific embodiments in which the subject matter may be practiced. Other embodiments may be utilized and derived therefrom, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is in fact disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A method for generating and updating one or more user interfaces of a mobile software application operating on a smart phone of a stroke survivor having an impairment, the method comprising:
tracking, from a plurality of network-based non-transitory storage devices, a first state of stroke survivor post discharge from a hospital;
generating a first user interface configured to be displayed on the smart phone, wherein the first user interface is dynamically updated based on the tracked first state of the stroke survivor;
including a first ribbon in the first user interface, said first ribbon corresponding to a first learning content enabled on the first user interface based on the first tracked state;
determining a plurality of first learning content user interfaces to split the learning content based on the impairment associated with the stroke survivor and a first property of the first learning content; and
including a second ribbon in the first user interface, said second ribbon corresponding to a second learning content, wherein the second ribbon is disabled until the plurality of first learning content user interfaces are viewed by the stroke survivor.

2. The method of claim 1, further comprising storing one or more metrics corresponding to the stroke survivor after one or more first learning content user interfaces are viewed by the stroke survivor, wherein the one or more metrics are stored in relation with a first electronic identification corresponding to the first learning content.

3. The method of claim 2, further comprising generating analysis based on the stored one or more metrics corresponding to a plurality of stroke survivors for the first learning content.

4. The method of claim 3, further comprising changing a timing of delivery of the first learning content to the mobile software application based on the generated analysis.

5. The method of claim 2, wherein the one or more metrics comprises a compliance measurement.

6. The method of claim 2, wherein the one or more metrics comprise a fall detection event.

7. The method of claim 2, wherein the one or metrics comprise an indication of infection.

8. The method of claim 1, wherein a first electronic identification is associated with the learning content.

9. The method of claim 8, further comprising removing the learning content from the mobile software application based on the first electronic identification.

10. The method of claim 1, further comprising providing a dashboard user interface, said dashboard interface comprising a plurality of tabs; and including a notification indicator adjacent to one of the plurality of tabs, said notification indicator corresponding to an activity detected from the mobile software application.

11. The method of claim 1, further comprising updating the first learning content based on one or more scores measured from an assessment.

12. The method of claim 1, further comprising generating a suggested list of questions based on the first state prior to an appointment with a health care team member.

13. The method of claim 12, further comprising providing an ability to digitally record an answer from the appointment.

14. The method of claim 1, further comprising enabling for a plurality of users, from a web interface, an ability to add ideas for a plurality of learning content, write a second learning content, review the second learning content, and send the second learning content to the mobile software application, without requiring the plurality of users to download any local copies corresponding to the learning content and without requiring opening of separate software applications.

15. A system for generating and updating one or more user interfaces of a mobile software application operating on a smart phone of a stroke survivor having an impairment, the system comprising one or more hardware processors configured to:
track, from a plurality of network-based non-transitory storage devices, a first state of stroke survivor post discharge from a hospital;
generate a first user interface configured to be displayed on the smart phone, wherein the first user interface is dynamically updated based on the tracked first state of the stroke survivor;
include a first ribbon in the first user interface, said first ribbon corresponding to a first learning content enabled on the first user interface based on the first tracked state;
determine a plurality of first learning content user interfaces to split the learning content based on the impairment associated with the stroke survivor and a first property of the first learning content; and
include a second ribbon in the first user interface, said second ribbon corresponding to a second learning content, wherein the second ribbon is disabled until the plurality of first learning content user interfaces are viewed by the stroke survivor.

16. The system of claim 15, wherein the one or more hardware processors are further configured to store one or more metrics corresponding to the stroke survivor after one or more first learning content user interfaces are viewed by the stroke survivor, wherein the one or more metrics are stored in relation with a first electronic identification corresponding to the first learning content.

17. The system of claim 16, wherein the one or more hardware processors are further configured to generate analysis based on the stored one or more metrics corresponding to a plurality of stroke survivors for the first learning content.

18. The system of claim 17, wherein the one or more hardware processors are further configured to change a timing of delivery of the first learning content to the mobile software application based on the generated analysis.

19. The system of claim 16, wherein the one or more metrics comprises a compliance measurement.

20. The system of claim 15, wherein the one or more hardware processors are further configured to enable for a plurality of users, from a web interface, an ability to add ideas for a plurality of learning content, write a second learning content, review the second learning content, and send the second learning content to the mobile software application, without requiring the plurality of users to download any local copies corresponding to the learning content and without requiring opening of separate software applications.
